(19) United States
(12) Patent Application Publication
(10) Pub. No.: US 2002/0006639 A1
(43) Pub. Date: Jan. 17, 2002

(54) Disease-associated gene

(76) Inventors: Paul Andrew Whittaker, West Sussex (GB); Stephen James Jones, West Sussex (GB); Marcus Thorold Hanley, West Sussex (GB)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/768,436

(22) Filed: Jan. 24, 2001

Related U.S. Application Data
(60) Provisional application No. 60/266,315, filed on Jan. 25, 2000.

(51) Int. Cl. A61K 48/00; C12Q 1/68; C07H 21/04; C12P 21/02

(52) U.S. Cl. 435/69.1; 435/6; 435/183; 435/325; 514/44; 536/23.2

(57) ABSTRACT

The invention provides an asthma-associated gene, the protein molecule encoded by the gene, the use of the gene in diagnosis, prognosis and treatment of inflammatory or obstructive airways diseases and use of the protein as a therapeutic target.

The present invention relates to a novel asthma-associated gene, designated AAG6, and to the protein molecule encoded by AAG6. The invention also relates to the use of AAG6 polynucleotide sequences for diagnostic and prognostic screening of patient populations and the use of the protein encoded by AAG6 as a therapeutic target.

Asthma is a very common lung disease with the following characteristics:

airways obstruction—this is usually reversible but often progressive

chronic bronchial inflammation—a condition characterised by inflammatory cell infiltration and activation, release of biochemical mediators and structural changes (airway remodelling)

bronchial hyperresponsiveness (BHR)—an exaggerated bronchoconstrictor response to a variety of immunologic, biochemical and physical stimuli.

Asthma is characterised clinically by chronic, intermittent airway obstruction with wheezing, coughing and breathlessness. Although asthma is typically associated with an obstructive impairment that is reversible, neither this finding nor any other single test or measure is adequate to diagnose asthma [Guidelines for the diagnosis and development of asthma, 1997, NIH Publication No. 97-4051]. Many diseases are associated with this pattern of abnormality. The patient's pattern of symptoms (along with other information from the patient's medical history) and exclusion of other possible diagnoses also are needed to establish a diagnosis of asthma. Clinical judgement is needed in conducting the assessment for asthma. Patients with asthma are heterogeneous and present signs and symptoms that vary widely from patient to patient as well as within each patient over time.

Many hypotheses have been advanced to explain the pathophysiology of asthma, including problems with airway smooth muscle, the role of inflammation, nervous innervation of the airways and mechanisms related to mediators. Although all of these factors may be important, it is unclear which are the primary (i.e. causative) defects and which are the secondary defects. It is generally agreed, however, that both the environment and genetics are important. Given the multifactorial nature of asthma, one approach to identifying the fundamental mechanisms is to discover asthma susceptibility genes that predispose individuals to develop asthma.

One method which can be used to identify asthma susceptibility genes is positional cloning. In this method, susceptibility genes are localised to a specific region of a human chromosome by using DNA markers to track the inheritance of the genes through families. DNA markers are fragments of DNA with a defined physical location on a chromosome, whose inheritance can be monitored. The closer a DNA marker is to a susceptibility gene, the greater the probability that the marker and the susceptibility gene will be passed together from parent to child. This phenomenon is called genetic linkage. Once linkage to a specific chromosomal region has been obtained, the size of the region is narrowed down using a combination of physical and genetic mapping until the region is small enough to be sequenced and the susceptibility gene can be identified. After identification of the susceptibility gene, any polymorphisms in this gene can be determined and an analysis performed to see whether these mutations occur with greater prevalence in asthmatics compared to non-asthmatics. The major advantages of positional cloning are that it is possible to identify novel genes even though the underlying factors causing the disease are unknown, and the genes identified are of direct pathological relevance (i.e. primary causative defects) because they make carriers directly susceptible to developing the disease.

In recent years a number of academic research groups have provided evidence for the presence of genes important in the regulation of asthmatic and allergic responses on human chromosome 5. In particular, evidence for the presence of susceptibility genes for BHR and elevated serum IgE levels on chromosome 5 in subregion 5q31-5q33 [Meyers et al., Genomics 23: 464-470; Postma et al., N. Eng. J. Med. 333:894-900; and Bleecker et al., Clin. Exp. Allergy 25:84-88] was obtained from genetic linkage analysis of 92 Dutch asthma families. Strong evidence for genetic linkage between marker D5S436, raised total serum IgE levels [Meyers et al., Genomics 23: 464-470; Postma et al., N. Eng. J. Med. 333:894-900; and Bleecker et al., Clin. Exp. Allergy 25:84-88] and BHR [Postma et al., N. Eng. J. Med. 333:894-900; and Bleecker et al., Clin. Exp. Allergy 25:84-88] was found in the Dutch families.

No asthma susceptibility gene has yet been identified, so there is a need in the art for the identification of such genes. Identification of asthma susceptibility genes would provide a fundamental understanding of the disease process from which a number of clinically important applications would arise. Susceptibility genes identified may lead to the development of therapeutics (small molecule drugs, antisense molecules, antibody molecules) directly targeted to the gene or protein product of the gene, or may target the biochemical pathway of which the protein product is a part at an upstream or downstream location if the development of such drugs is easier than directly targeting the gene or its protein product. Polynucleotide sequences comprising the gene, sequence variants thereof and protein products thereof may be used to develop a clinical diagnostic test for asthma and for the identification of individuals at high risk for the development of asthma. The results of such tests may also have prognostic value and may be used to predict patients who respond to and those who do not respond to drug therapy. Finally, information about the DNA sequences of asthma susceptibility genes and the amino acid sequences encoded by these genes facilitates large scale production of proteins by recombinant techniques and identification of the tissues/cells naturally producing the proteins. Such sequence information also permits the preparation of antibody substances or other novel binding molecules specifically reactive with the proteins encoded by the susceptibility genes that may be used in modulating the natural ligand/antiligand binding reactions in which the proteins may be involved and for diagnostic purposes.

Accordingly, the present invention provides, in one aspect, an isolated polynucleotide, hereinafter alternatively referred to as AAG6, comprising a nucleotide sequence encoding a polypeptide comprising the amino acid sequence of SEQ ID NO:2 or SEQ ID NO:4 or a functionally equivalent variant of said amino acid sequence, i.e. a variant thereof which retains the biological or other functional activity thereof, e.g. a variant which is capable of raising an antibody which binds to a polypeptide comprising the amino acid sequence of SEQ ID NO:2 or SEQ ID NO:4.

Terms used herein have the following meanings:

“Isolated” refers to material removed from its original environment.

“Hybridization” or “hybridizes” refers to any process by which a strand of a polynucleotide binds with a complementary strand through base pairing.

“Stringent conditions” refer to experimental conditions which allow up to 20% base pair mismatches, typically two 15 minute washes in 0.1×SSC (15mM NaCl, 1.5 mM sodium citrate, pH 7.0) at 65° C. “Homology” or “homologous” refers to a degree of similarity between nucleotide or amino acid sequences, which may be partial or, when sequences are identical, complete.

“Expression vector” refers to a linear or circular DNA molecule which comprises a segment encoding a polypeptide of interest operably linked to additional segments which provide for its transcription.

“Antisense” refers to selective inhibition of protein synthesis through hybridisation of an oligo- or polynucleotide to its complementary sequence in messenger RNA (mRNA) of the target protein. The antisense concept was first proposed by Zamecnik and Stephenson (Proc. Natl. Acad. Sci. USA 75:280-284; Proc. Natl. Acad. Sci. USA 75:285-288) and has subsequently found broad application both as an experimental tool and as a means of generating putative therapeutic molecules (Alama, A., Pharmacol. Res. 36:171-178; Dean, N. M., Biochem. Soc. Trans. 24:623-629; Bennet, C. F., J. Pharmacol. Exp. Ther. 280:988-1000; Crooke, S. T., Antisense Research and Applications, Springer).

The term “variant” as used herein means, in relation to amino acid sequences, an amino acid sequence that is altered by one or more amino acids. The changes may involve amino acid substitution, deletion or insertion. In relation to nucleotide sequences, the term “variant” as used herein means a nucleotide sequence that is altered by one or more nucleotides; the changes may involve nucleotide substitution, deletion or insertion. A preferred functionally equivalent variant of the amino acid sequence SEQ ID NO:2 or SEQ ID NO:4 is one having at least 80%, more preferably at least 90%, and especially more than 95% amino acid sequence identity to SEQ ID NO:2 or SEQ ID NO:4. In such preferred functionally equivalent variants, the regions of SEQ ID NO:2 (amino acids 1-334) or SEQ ID NO:4 (amino acids 1-710) corresponding to the extracellular domain are usually substantially conserved.

By an amino acid sequence having x% identity to a reference sequence such as SEQ ID NO:2 or SEQ ID NO:4, is meant a sequence which is identical to the reference sequence except that it may include up to 100-x amino acid alterations per each 100 amino acids of the reference sequence. For example, in a subject amino acid sequence having at least 80% identity to a reference sequence, up to 20% of the amino acid residues in the reference sequence may be substituted, deleted or inserted with another amino acid residue. Percentage identity between amino acid sequences can be determined conventionally using known computer programs, for example the FASTDB program based on the algorithm of Brutlag et al (Comp.App.Biosci. (1990) 6:237-245).

The isolated polynucleotide of the invention may be cDNA, genomic DNA or RNA. In particular embodiments, the isolated polynucleotide is cDNA comprising the nucleotide sequence of SEQ ID NO:1 or SEQ ID NO:3, a genomic DNA comprising the nucleotide sequence of SEQ ID NO:5 or a DNA comprising a nucleotide sequence which hybridises to SEQ ID NO:1 or SEQ ID NO:3 or SEQ ID NO:5 under stringent conditions.

The invention also provides an isolated polynucleotide comprising a consecutive 20 base pair nucleotide portion identical in sequence to a consecutive 20 base pair portion of SEQ ID NO:1 or SEQ ID NO:3 or SEQ ID NO:5.

A polynucleotide of the invention may be isolated by bioinformatics analysis of DNA sequences from the subregion 5q31-5q33 on chromosome 5 determined by sequencing of yeast artificial chromosomes (YACs), bacterial artificial chromosomes (BACs) and/or P1 artificial chromosomes (PACs) to identify genes within that subregion, searching for a sequence having greater than 95% identity to the predicted exon for a selected gene and isolating cDNA from a human lung cDNA library by PCR using primers designed using that sequence.

A polynucleotide of the invention, for example having the sequence SEQ ID NO:1 or SEQ ID NO:3 may be prepared from the nucleotides which it comprises by chemical synthesis, e.g. automated solid phase synthesis using known procedures and apparatus.

In another aspect, the present invention provides an isolated polypeptide, particularly a recombinant polypeptide, comprising the amino acid sequence of SEQ ID NO:2, or SEQ ID NO:4 or a functionally equivalent variant thereof. Such a polypeptide may be produced by cloning a polynucleotide sequence as hereinbefore described into an expression vector containing a promoter and other appropriate regulating elements for transcription, transferring into prokaryotic or eukaryotic host cells such as bacterial, plant, insect, yeast, animal or human cells, and culturing the host cells containing the recombinant expression vector under suitable conditions. Techniques for such recombinant expression of polypeptides are well known and are described, for example, in J. Sambrook et al, Molecular Cloning, second edition, Cold Spring Harbor Press, 1990. The polypeptide of the invention, i.e. the polypeptide encoded by the AAG6 polynucleotide of the invention, has high homology to cadherin proteins, which are important participants in cell-cell adhesion-see M. Takeichi, Annu. Rev. Biochem (1990), 58, 237-52.

Accordingly, the present invention also provides a method of producing a polypeptide of the invention which comprises culturing a host cell containing an expression vector containing a polynucleotide sequence of the invention as hereinbefore described under conditions suitable for expression of the polypeptide and recovering the polypeptide from the host cell culture.

In another aspect, the present invention provides an expression vector containing a polynucleotide sequence of the invention as hereinbefore described.

The invention also provides an isolated polypeptide comprising a consecutive 10 amino acid portion identical in sequence to a consecutive 10 amino acid portion of SEQ ID NO:2, or SEQ ID NO:4.

A polypeptide of the invention may be expressed as a recombinant fusion protein with one or more heterologous polypeptides, for example to facilitate purification. For example, it may be expressed as a recombinant fusion protein with a heterologous polypeptide such as a polyhistidine containing a cleavage site located between the polynucleotide sequence of the invention and the heterologous polypeptide sequence, so that the polypeptide comprising the amino acid sequence of SEQ ID NO:2 or SEQ ID NO:4 may be cleaved and purified away from the heterologous moiety using well known techniques.

A polypeptide of the invention may also be synthesised, in whole or in part, from the amino acids which it comprises using well known chemical methods, for example automated solid phase techniques.

Isolated polypeptides of the invention as hereinbefore described may be purified by well known standard procedures.

The present invention also provides an antibody which is immunoreactive with a polypeptide of the invention as hereinbefore described, or a variant of said polypeptide having a polymorphism correlated with a particular disease, e.g. asthma. The antibody may be a polyclonal or monoclonal antibody. Such antibodies may be prepared using conventional procedures. Methods for the production of polyclonal antibodies against purified antigen are well established (cf. Cooper and Paterson in Current Protocols in Molecular Biology, Ausubel et al. Eds., John Wiley and Sons Inc., Chapter 11). Typically, a host animal, such as a rabbit, or a mouse, is immunised with a purified polypeptide of the invention, or immunogenic portion thereof, as antigen and, following an appropriate time interval, the host serum is collected and tested for antibodies specific against the polypeptide. Methods for the production of monoclonal antibodies against purified antigen are well established (cf. Chapter 11, Current Protocols in Molecular Biology, Ausubel et al. Eds., John Wiley and Sons Inc.). For the production of a polyclonal antibody, the serum can be treated with saturated ammonium sulphate or DEAE Sephadex. For the production of a monoclonal antibody, the spleen or lymphocytes of the immunised animal are removed and immortalised or used to produce hybridomas by known methods. Antibodies secreted by the immortalised cells are screened to determine the clones which secrete antibodies of the desired specificity, for example using Western blot analysis. Humanised antibodies can be prepared by conventional procedures.

In another aspect, the present invention provides an antisense oligonucleotide comprising a nucleotide sequence complementary to that of a polynucleotide of the invention, in particular a nucleotide sequence complementary to SEQ ID NO:1 or SEQ ID NO:3 or SEQ ID NO:5, or complementary to that of a polynucleotide encoding a variant of a polypeptide of the invention having a polymorphism correlated with a disease, e.g. asthma, in particular a nucleotide sequence complementary to such a polymorphic variant of SEQ ID NO:1 or SEQ ID NO:3 or SEQ ID NO:5. The antisense oligonucleotide may be DNA, an analogue of DNA such as a phosphorothioate or methylphosphonate analogue of DNA, RNA, an analogue of RNA, or a peptide nucleic acid (PNA). The antisense oligonucleotides may be synthesised by conventional methods, for example using automated solid phase techniques.

The present invention also provides a polynucleotide probe comprising at least 15 contiguous nucleotides of a polynucleotide of the invention or a complement thereof. The probe may be cDNA, genomic DNA or RNA. Usually it is a synthetic oligonucleotide comprising 15 to 50 nucleotides, which can be labelled, e.g. with a fluorophore, to provide a detectable signal.

The polynucleotide probe is capable of selectively hybridising under stringent conditions to a polynucleotide fragment having a sequence selected from the group consisting of SEQ IDS: 1, 3 and 5. The probe has a sequence such that under such hybridisation conditions it hybridizes only to its cognate sequence. DNA probes as described above are useful in a number of screening applications including Northern and Southern blot analyses, dot blot and slot blot analyses, and fluorescence in situ hybridisation (FISH).

The present invention also includes a pair of oligonucleotides having nucleotide sequences useful as primers for DNA amplification of a fragment of a polynucleotide of the invention, i.e. of the human AAG6 gene (hAAG6), wherein each primer of said pair is at least 15 nucleotides in length and said pair have sequences such that when used in a polymerase chain reaction (PCR) with either human genomic DNA or a suitable human cDNA target they result in synthesis of a DNA fragment containing all or preferably part of the sequence of hAAG6. The primer pair is preferably capable of amplifying at least one exon of hAAG6 (or portion thereof), such as an exon selected from those in SEQ ID NO:1, 3 or 5. Examples of such primer pairs are shown hereinafter in the Examples. Exemplary applications of such primer pairs include amplification of DNA fragments for use in the detection of changes to the polynucleotide sequence in asthmatic patients as shown hereinafter in the Examples.

The role of the polypeptide of the invention in asthma and other obstructive or inflammatory airways diseases characterised by bronchial hyperresponsiveness can be determined using conventional allergen driven animal models for bronchial hyperresponsiveness, e.g. the ovalbumin-induced BHR mouse model (Tsuyuki et al, J. Clin. Invest. 96:2924-2931) or the guinea pig model hereinafter described.

Polynucleotides, polypeptides, antibodies, antisense oligonucleotides or probes of the invention as hereinbefore described, hereinafter alternatively referred to collectively as agents of the invention, may be used in the treatment (prophylactic or symptomatic) or diagnosis of inflammatory or obstructive airways diseases. For example, a polypeptide of the invention may be used to treat a mammal, particularly a human, deficient in or otherwise in need of that polypeptide; a polynucleotide of the invention may be used in gene therapy where it is desired to increase AAG6 activity, for instance where a subject has a mutated or missing AAG6 gene; an antisense oligonucleotide of the invention may be used to inhibit AAG6 activity or activity of variants of the AAG6 gene having a polymorphism correlated with a disease, e.g. asthma, where this is desired; an antibody of the invention may be used to detect, or determine the level of expression of, AAG6 polypeptides or a disease-correlated polymorphic variant thereof, or to inhibit ligand/antiligand binding activities of AAG6 polypeptides; and a probe of the invention may be used to detect the presence or absence of the AAG6 gene, i.e. to detect genetic abnormality.

“Gene therapy” refers to an approach to the treatment of human disease based upon the transfer of genetic material into somatic cells of an individual. Gene transfer can be achieved directly in vivo by administration of gene-bearing viral or non-viral vectors into blood or tissues, or indirectly ex vivo through the introduction of genetic material into cells manipulated in the laboratory followed by delivery of the gene-containing cells back to the individual. By altering the genetic material within a cell, gene therapy may correct underlying disease pathophysiology. Suitable vectors, and procedures, for gene delivery to specific tissues and organ systems in animals are described in Dracopoli, N. C. et al., Current Protocols in Human Genetics. John Wiley and Sons Inc., Chapters 12 and 13 respectively. In relation to polynucleotides of the invention, gene therapy may involve delivery of a viral or non-viral gene therapy vector containing an expression cassette of the AAG6 gene under suitable control elements to the lungs of diseased individuals (eg. asthmatics) so that the underlying disease pathophysiology is corrected or ameliorated.

Accordingly, in further aspects, the present invention provides

a pharmaceutical composition comprising a polynucleotide, polypeptide, antibody or antisense oligonucleotide of the invention as hereinbefore described, optionally together with a pharmaceutically acceptable carrier;

a method of treating an inflammatory or obstructive airways disease which comprises administering to a subject in need thereof an effective amount of a polynucleotide, polypeptide, antibody or antisense oligonucleotide of the invention as hereinbefore described;

a method of detecting genetic abnormality in a subject which comprises incubating a genetic sample from the subject with a polynucleotide probe of the invention as hereinbefore defined, under conditions where the probe hybridises to complementary polynucleotide sequence, to produce a first reaction product, and comparing the first reaction product to a control reaction product obtained with a normal genetic sample, where a difference between the first reaction product and the control reaction product indicates a genetic abnormality in the subject or a predisposition to developing a disease such as asthma;

a method of detecting the presence of a polynucleotide of the invention, e.g. comprising SEQ ID NO:1, 3 or 5, in cells or tissues which comprises contacting DNA from the cell or tissue with a polynucleotide probe as hereinbefore defined under conditions where the probe is specifically hybridizable with a polynucleotide of the invention, and detecting whether hybridization occurs;

a method of detecting an abnormality in the nucleotide sequence of a polynucleotide of the invention in a patient which comprises amplifying a target nucleotide sequence in DNA isolated from the patient by a polymerase chain reaction using a pair of primers as hereinbefore described which target the sequence to be amplified and analysing the amplified sequence to determine any polymorphism present therein; and

a method of detecting polymorphism in a subject which comprises treating a tissue sample from the subject with an antibody to a polymorphic variant of a polypeptide of the invention and detecting binding of said antibody.

The term “polymorphism” means any sequence difference as compared with the sequence of a polynucleotide of the invention as hereinbefore described.

Information obtained using the diagnostic assays described herein (alone or in conjunction with information on another genetic defect, which contributes to the same disease) is useful for prognosing, diagnosing or confirming that a symptomatic subject has a genetic defect (e.g. in an AAG6 gene or in a gene that regulates the expression of an AAG6 gene), which causes or contributes to the particular disease or disorder. Alternatively, the information (alone or in conjunction with information on another genetic defect, which contributes to the same disease) can be used prognostically for predicting whether a non-symptomatic subject is likely to develop a disease or condition, which is caused by or contributed to by an abnormal AAG6 activity or protein level in a subject. In particular, the assays permit one to ascertain an individual's predilection to develop a condition associated with a mutation in or associated with AAG6, where the mutation is a polymorphism such as a single nucleotide polymorphism (SNP). Based on the prognostic information, a doctor can recommend a regimen e.g. a therapeutic protocol useful for preventing or delaying onset of asthma in the individual.

In addition, knowledge of the particular alteration or alterations, resulting in defective or deficient AAG6 genes or proteins in an individual, alone or in conjunction with information on other genetic defects contributing to the same disease (the genetic profile of the particular disease) allows customization of therapy to the individual's genetic profile, the goal of pharmacogenomics. For example, an individual's AAG6 genetic profile or the genetic profile of the asthma can enable a doctor to: 1) more effectively prescribe a drug that will address the molecular basis of asthma; and 2) better determine the appropriate dosage of a particular drug. For example, the expression level of AAG6 proteins, alone or in conjunction with the expression level of other genes known to be involved in asthma, can be measured in many patients at various stages of the disease to generate a transcriptional or expression profile of asthma. Expression patterns of individual patients can then be compared to the expression profile of asthma to determine the appropriate drug and dose to administer to the patient.

The ability to target populations expected to show the highest clinical benefit, based on the AAG6 or asthma genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since the use of AAG6 as a marker is useful for optimizing effective dose).

Hybridisation of a polynucleotide probe of the invention with complementary polynucleotide sequence may be detected using in situ (eg. FISH) hybridization, Northern or Southern blot analyses, dot blot or slot blot analyses. The abnormality may also be detected for example by conformation sensitive gel electrophoresis (CSGE) and DNA sequencing as described hereinafter in the Examples. The genetic abnormality may result in a change in the amino acid sequence of the individual's AAG6 protein relative to the the amino acid sequence of a normal hAAG6 protein, or loss of protein. Alternatively, the change may not alter the amino acid sequence but may instead alter expression of the AAG6 gene by altering the sequence of controlling elements either at the 5'-, or 3'-end of the gene, or altering the sequence of control elements within intronic regions of the gene. Changes may also affect the way the gene transcript is processed or translated. The invention also includes kits for the detection of an abnormality in the polynucleotide sequence of an individual's AAG6 gene. Hybridisation kits for such detection comprise a probe of the invention as hereinbefore described, which probe may be modified by incorporation of a detectable, e.g. chemiluminescent or fluorescent, label therein, and may include other reagents such as labelling reagents, i.e. reagents to incorporate a detectable label such as a radioactive isotope, chemiluminescent or fluorescent group into a hybridised product, and buffers. PCR amplification kits comprise primer pairs such as those described above together with a DNA polymerase such as Taq polymerase, and may include additional reagents, such as an amplification buffer and the like. Specific embodiments of the PCR amplification kits can include additional reagents specific for a number of techniques that detect polynucleotide changes, including CSGE and DNA sequencing.

The effectiveness of an agent of the invention in inhibiting or reversing airways hyperreactivity may be demonstrated in a guinea pig test model. The acute injection of preformed immune complex renders guinea pigs hyperreactive to histamine. Doses of histamine which cause only a small degree of bronchoconstriction prior to administration of immune complex cause a much stronger effect thereafter. Guinea-pigs (Dunkin-Hartley, male, 400-600 g) are anaesthetised with phenobarbital (100 mg/kg i.p.) and pentobarbital (30 mg/kg i.p.) and paralysed with gallamine (10 mg/kg i.m.) and ventilated with a mixture of air and oxygen (45:55), v/v). Animals are ventilated (8 ml/kg, 1 Hz) via a tracheal cannula. Ventilation is monitored by a flow transducer. When making measurements of flow, coincident pressure changes in the thorax are monitored directly via an intrathoracic trochar, permitting display of differential pressure relative to the trachea. From this information resistance and compliance are calculated at each inspiration. An allergic reaction is initiated by intravenous injection of preformed immune complexes (prepared by adding 30 μg of bovine gamma globulin in 0.05 ml of saline to 0.05 ml of guinea pig anti-bovine gamma globulin anti-serum) 3 times at 10 minute intervals. Intravenous injections of histamine (1.0-3.2 μg/kg at 10 minute intervals) are used to define the sensitivity of the airways prior to and following the last exposure to the immune complex. Airways hyperreactivity is expressed as the paired difference for the maximal value of lung resistance in response to histamine before and after repeated injection of immune-complex. The agents of the invention are administered intratracheally either as solutions or suspensions in tragacanth. The ED₅₀ values for reversal of airways hyperreactivity are determined graphically from the dose response curves and represent those doses which cause a 50% reduction of airways hyperreactivity.

Inflammatory or obstructive airways diseases to which the present invention is applicable include asthma of whatever type or genesis including both intrinsic (non-allergic) asthma and extrinsic (allergic) asthma. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or reduced airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include adult respiratory distress syndrome (ARDS), chronic obstructive pulmonary or airways disease (COPD or COAD), including chronic bronchitis, or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, agents of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

The agents of the invention may be administered by any appropriate route, e.g. orally, for example in the form of a tablet or capsule; parenterally, for example intravenously; topically, e.g. in an ointment or cream; transdermally, e.g. in a patch; by inhalation; or intranasally.

Pharmaceutical compositions containing agents of the invention may be prepared using conventional diluents or excipients and techniques known in the galenic art. Thus oral dosage forms may include tablets and capsules, and compositions for inhalation may comprise aerosol or other atomizable formulations or dry powder formulations.

The invention includes (A) an agent of the invention in inhalable form, e.g. in an aerosol or other atomizable composition or in inhalable particulate, e.g. micronised form, (B) an inhalable medicament comprising an agent of the invention in inhalable form; (C) a pharmaceutical product comprising such an agent of the invention in inhalable form in association with an inhalation device; and (D) an inhalation device containing an agent of the invention in inhalable form.

Dosages of agents of the invention employed in practising the present invention will of course vary depending, for example, on the particular condition to be treated, the effect desired and the mode of administration. In general, suitable daily dosages for administration by inhalation are of the order of 1μg to 10 mg/kg while for oral administration suitable daily doses are of the order of 0.1 mg to 1000 mg/kg.

The present invention also provides a variant of a polynucleotide of the invention as hereinbefore described, particularly a polynucleotide having a sequence SEQ ID NO:1 or SEQ ID NO:3 or SEQ ID NO:5, which contains a sequence polymorphism correlated with asthma. The polymorphism may be an addition, deletion or replacement of one or more nucleotides. Single nucleotide polymorphisms (SNPs), as compared with SEQ ID NO:1 or SEQ ID NO:3, or SEQ ID NO:5, which have been identified in genetic samples from asthmatic patients are shown hereinafter in the Examples.

The present invention further provides a method of determining predisposition of a subject to asthma comprising determining the presence or absence in DNA from the subject of a sequence polymorphism in a polynucleotide of the invention which correlates with asthma.

In another aspect, the present invention provides a method of determining predisposition of a patient to asthma which comprises identifying in DNA from the patient a sequence polymorphism or haplotype in a polynucleotide of the invention, as compared with a normal control DNA from a non-asthmatic subject, which correlates with asthma. A haplotype is a set of polymorphisms which is inherited together as a group.

In a related aspect, the invention provides a method of determining predisposition of a patient to asthma which comprises identifying in DNA from the patient a sequence polymorphism or haplotype in a polynucleotide of the invention, as compared with SEQ ID NO:1 or SEQ ID NO:3 or SEQ ID NO:5 which correlates with asthma.

Identification of a sequence polymorphism may be effected by conventional sequencing and sequence analysis techniques, for example as described in Cotton, R. G. H., Mutation Detection, Oxford University Press, 1997; Landegren, U., Laboratory Protocols for Mutation Detection, Oxford University Press; and R. G. H. Cotton et al, Mutation Detection, Oxford University Press, 1998.

Sequence polymorphisms which correlate with asthma may alter the amino acid sequence in the encoded polypeptide or may affect expression levels of the polypeptide or the way in which a transcript is processed.

Certain sequence polymorphisms or haplotypes may correlate with the severity and/or nature of the asthmatic phenotype, e.g. with mild, moderate or severe asthma as defined by established clinical parameters. Identification of polymorphisms may therefore be useful for prognosis, determination of therapeutic strategy and prediction of patient responses to therapy.

In particular, the invention further features predictive medicines, which are based, at least in part, on the identity of the novel AAG6 gene and alterations in the genes and related pathway genes, which affect the expression level and/or function of the encoded AAG6 protein in a subject.

For example, as described herein, AAG6 mutations that are particularly likely to cause or contribute to the development of asthma or other inflammatory or obstructive airways diseases characterised by BHR are those mutations that negatively impact normal (wildtype) functioning of AAG6, in particular the extracellular domain which is involved in homotypic association and therefore cell-cell adhesion and the intracellular domain which interacts with structural proteins or signalling molecules. Examples of such mutations include: i) mutations that affect the level of transcripts produced; ii) missense mutations occurring within the intracellular, transmembrane or extracellular domain; and mutations which affect the way in which the transcript is processed.

The present methods provide means for determining if a subject has (diagnostic) or is at risk of developing (prognostic) a disease, condition or disorder that is associated with an aberrant AAG6 activity, e.g., an aberrant level of AAG6 protein or an aberrant bioactivity, such as results in the development of asthma.

Accordingly, the invention provides methods for determining whether a subject has or is likely to develop an obstructive or inflammatory airways disease such as asthma, comprising determining the level of an AAG6 gene or protein, an AAG6 bioactivity and/or the presence of a mutation or particular polymorphic variant in the AAG6 gene.

In one embodiment, the method comprises determining whether a subject has an abnormal mRNA and/or protein level of AAG6, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the AAG6 protein or mRNA level is determined and compared to the level of AAG6 protein or mRNA level in a healthy subject. An abnormal level of AAG6 polypeptide or mRNA level is likely to be indicative of an aberrant AAG6 activity.

In another embodiment, the method comprises measuring at least one activity of AAG6. For example, calcium-dependent cell-cell adhesion can be measured, e.g. as described herein. Comparison of the results obtained with results from similar analysis performed on AAG6 proteins from healthy subjects is indicative of whether a subject has an abnormal AAG6 activity.

In preferred embodiments, the methods for determining whether a subject has or is at risk for developing a disease, which is caused by or contributed to by an aberrant AAG6 activity is characterized as comprising detecting, in a sample of cells from the subject, the presence or absence of a genetic alteration characterized by at least one of: (i) an alteration affecting the integrity of a gene encoding an AAG6 polypeptide, or (ii) the mis-expression of the AAG6 gene. For example, such genetic alterations can be detected by ascertaining the existence of at least one of: (i) a deletion of one or more nucleotides from an AAG6 gene, (ii) an addition of one or more nucleotides to an AAG6 gene, (iii) a substitution of one or more nucleotides of an AAG6 gene, (iv) a gross chromosomal rearrangement of an AAG6 gene, (v) a gross alteration in the level of a messenger RNA transcript of an AAG6 gene, (vi) aberrant modification of an AAG6 gene, such as of the methylation pattern of the genomic DNA, (vii) the presence of a non-wild type splicing pattern of a messenger RNA transcript of an AAG6 gene, (viii) a non-wild type level of an AAG6 polypeptide, (ix) allelic loss of an AAG6 gene, and/or (x) inappropriate post-translational modification of an AAG6 polypeptide. The present invention provides a large number of assay techniques for detecting alterations in an AAG6 gene. These methods include, but are not limited to, methods involving sequence analysis, Southern blot hybridization, restriction enzyme site mapping, and methods involving detection of the absence of nucleotide pairing between the nucleic acid to be analyzed and a probe.

Specific diseases or disorders, e.g., genetic diseases or disorders, are associated with specific allelic variants of polymorphic regions of certain genes, which do not necessarily encode a mutated protein. Thus, the presence of a specific allelic variant of a polymorphic region of a gene, such as a single nucleotide polymorphism (“SNP”), in a subject can render the subject susceptible to developing a specific disease or disorder. Polymorphic regions in genes, e.g., AAG6 genes, can be identified, by determining the nucleotide sequence of genes in populations of individuals. If a polymorphic region, e.g., SNP is identified, then the link with a specific disease can be determined by studying specific populations of individuals, e.g., individuals which developed a specific disease, such as asthma. A polymorphic region can be located in any region of a gene, e.g., exons, in coding or non coding regions of exons, introns, and promoter region.

It is likely that AAG6 genes comprise polymorphic regions, specific alleles of which may be associated with specific diseases or conditions or with an increased likelihood of developing such diseases or conditions. Thus, the invention provides methods for determining the identity of the allele or allelic variant of a polymorphic region of an AAG6 gene in a subject, to thereby determine whether the subject has or is at risk of developing a disease or disorder that is associated with a specific allelic variant of a polymorphic region.

In an exemplary embodiment, there is provided a nucleic acid composition comprising a nucleic acid probe including a region of nucleotide sequence which is capable of hybridizing to a sense or antisense sequence of an AAG6 gene or naturally occurring mutants thereof, or 5′ or 3′ flanking sequences naturally associated with the subject AAG6 genes or naturally occurring mutants thereof. The nucleic acid of a cell is rendered accessible for hybridization, the probe is contacted with the nucleic acid of the sample, and the hybridization of the probe to the sample nucleic acid is detected. Such techniques can be used to detect alterations or allelic variants at either the genomic or mRNA level, including deletions, substitutions, etc., as well as to determine mRNA transcript levels.

A preferred detection method is allele specific hybridization using probes overlapping the mutation or polymorphic site and having about 5, 10, 20, 25, or 30 nucleotides around the mutation or polymorphic region. In a preferred embodiment of the invention, several probes capable of hybridizing specifically to allelic variants, such as single nucleotide polymorphisms, are attached to a solid phase support, e.g., a “chip”. Oligonucleotides can be bound to a solid support by a variety of processes, including lithography. For example a chip can hold up to about 250,000 oligonucleotides. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In certain embodiments, detection of the alteration comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligase chain reaction (LCR) (see, e.g., Landegran et al. (1988) Science 241:1077-1080; and Nakazawa et al. (1994) PNAS 91:360-364), the latter of which can be particularly useful for detecting point mutations in the AAG6 gene (see Abravaya et al. (1995) Nuc Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to an AAG6 gene under conditions such that hybridization and amplification of the AAG6 gene (if present) occurs, and (iv) detecting the presence or absence of an amplification product, or detecting the size of the amplification product and comparing the length to a control sample. It is anticipated that PCR, LCR or any other amplification procedure (e.g. self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), or Q-Beta Replicase (Lizardi, P. M. et al., 1988, Bio/Technology 6:1197)), may be used as a preliminary step to increase the amount of sample on which can be performed any of the techniques for detecting mutations described herein.

Knowledge of the particular alteration or alterations, resulting in defective or deficient AAG6 genes or proteins in an individual (the AAG6 genetic profile), alone or in conjunction with information on other genetic defects contributing to the same disease (the genetic profile of the particular disease) allows a customization of the therapy for a particular disease to the individual's genetic profile, the goal of “pharmacogenomics”. For example, subjects having a specific allele of an AAG6 gene may or may not exhibit symptoms of a particular disease or be predisposed of developing symptoms of a particular disease. Further, if those subjects are symptomatic, they may or may not respond to a certain drug, e.g., a specific AAG6 therapeutic, but may respond to another. Thus, generation of an AAG6 genetic profile, (e.g., categorization of alterations in AAG6 genes which are associated with the development of asthma), from a population of subjects, who are symptomatic for a disease or condition that is caused by or contributed to by a defective and/or deficient AAG6 gene and/or protein (an AAG6 genetic population profile) and comparison of an individual's AAG6 profile to the population profile, permits the selection or design of drugs that are expected to be safe and efficacious for a particular patient or patient population (i.e., a group of patients having the same genetic alteration).

For example, an AAG6 population profile can be performed, by determining the AAG6 profile, e.g., the identity of AAG6 genes, in a patient population having a disease, which is caused by or contributed to by a defective or deficient AAG6 gene. Optionally, the AAG6 population profile can further include information relating to the response of the population to an AAG6 therapeutic, using any of a variety of methods, including, monitoring: 1) the severity of symptoms associated with the AAG6 related disease, 2) AAG6 gene expression level, 3) AAG6 mRNA level, and/or 4) AAG6 protein level. and (iii) dividing or categorizing the population based on the particular genetic alteration or alterations present in its AAG6 gene or an AAG6 pathway gene. The AAG6 genetic population profile can also, optionally, indicate those particular alterations in which the patient was either responsive or non-responsive to a particular therapeutic. This information or population profile, is then useful for predicting which individuals should respond to particular drugs, based on their individual AAG6 profile.

In a preferred embodiment, the AAG6 profile is a transcriptional or expression level profile and step (i) is comprised of determining the expression level of AAG6 proteins, alone or in conjunction with the expression level of other genes, known to contribute to the same disease. The AAG6 profile can be measured in many patients at various stages of the disease.

Pharmacogenomic studies can also be performed using transgenic animals. For example, one can produce transgenic mice, which contain a specific allelic variant of an AAG6 gene. These mice can be created, e.g., by replacing their wild-type AAG6 gene with an allele of the human AAG6 gene. The response of these mice to specific AAG6 therapeutics can then be determined.

The treatment of an individual with an AAG6 therapeutic can be monitored by determining AAG6 characteristics, such as AAG6 protein level or activity, AAG6 mRNA level, and/or AAG6 transcriptional level. These measurements will indicate whether the treatment is effective or whether it should be adjusted or optimized. Thus, AAG6 can be used as a marker for the efficacy of a drug during clinical trials.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a preadministration sample from a subject prior to administration of the agent; (ii) detecting the level of expression of an AAG6 protein, mRNA, or genomic DNA in the preadministration sample; (iii) obtaining one or more post-administration samples from the subject; (iv) detecting the level of expression or activity of the AAG6 protein, mRNA, or genomic DNA in the post-administration samples; (v) comparing the level of expression or activity of the AAG6 protein, mRNA, or genomic DNA in the preadministration sample with the AAG6 protein, mRNA, or genomic DNA in the post administration sample or samples; and (vi) altering the administration of the agent to the subject accordingly. For example, increased administration of the agent may be desirable to increase the expression or activity of AAG6 to higher levels than detected, i.e., to increase the effectiveness of the agent. Alternatively, decreased administration of the agent may be desirable to decrease expression or activity of AAG6 to lower levels than detected, i.e., to decrease the effectiveness of the agent.

Cells of a subject may also be obtained before and after administration of an AAG6 therapeutic to detect the level of expression of genes other than AAG6, to verify that the AAG6 therapeutic does not increase or decrease the expression of genes which could be deleterious. This can be done, e.g., by using the method of transcriptional profiling. Thus, mRNA from cells exposed in vivo to an AAG6 therapeutic and mRNA from the same type of cells that were not exposed to the AAG6 therapeutic could be reverse transcribed and hybridized to a chip containing DNA from numerous genes, to compare thereby the expression of genes in cells treated and not treated with an AAG6-therapeutic. If, for example an AAG6 therapeutic turns on the expression of a proto-oncogene in an individual, use of this particular AAG6 therapeutic may be undesirable.

A polypeptide of the invention, including a polypeptide encoded by a variant of a polynucleotide comprising a nucleotide sequence encoding a polypeptide comprising amino acid sequence SEQ ID NO:2 or SEQ ID NO:4, which variant contains a sequence polymorphism, can be used to identify enhancers (agonists) or inhibitors (antagonists) of its activity, i.e. to identify compounds useful in the treatment of inflammatory or obstructive airways diseases, particularly asthma. Accordingly, the invention also provides a method of identifying a substance which modulates the activity of a polypeptide of the invention comprising combining a candidate substance with a polypeptide of the invention and measuring the effect of the candidate substance on said activity. The activity of a polypeptide of the invention may be measured, for example, by promotion of homotypic Ca²⁺ dependent aggregation and adhesion in Chinese hamster ovary transfectants e.g. as described by Telo et al, J. Biol. Chem. 273:17565-17572. The invention also includes a method of identifying a substance which binds to a polypeptide of the invention as hereinbefore described comprising mixing a candidate substance with a polypeptide of the invention and determining whether binding has occurred.

The invention is illustrated by the following Examples. Abbreviations used in the Examples have the following meanings:

| | |
|---|---|
| AEBSF: | 4-(2-aminoethyl)benzenesulfonyl fluoride |
| BAC: | bacterial artificial chromosome |
| BAP: | 1,4-bis(acryloyl)piperazine |
| BLAST: | basic local alignment search tool |
| BSA: | bovine serum albumin |
| CSGE: | conformation sensitive gel electrophoresis |
| DNTP: | deoxynucleotide triphosphate |
| DTT: | dithiothreitol |
| EIA: | enzyme immunoassay |
| EST: | expressed sequence tag |
| FCS: | fetal calf serum |
| HUVEC: | human umbilical vein endothelial cell |
| MTN: | multiple tissue northern |
| MVEC2: | mouse vascular endothelial cadherin 2 |
| NHBE: | normal human bronchial epithelial |
| ORF: | open reading frame |
| PAC: | P1 artificial chromosome |
| PBS: | phosphate buffered saline |
| PEG: | polyethylene glycol |
| PMSF: | phenylmethylsulfonyl fluoride |
| SDS-PAGE: | sodium dodecyl sulfate polyacrylamide gel |
| | electrophoresis |
| SNP: | single nucleotide polymorphism |
| STS: | sequence tagged site |
| TIGR: | The Institute for Genome Research |
| TTE: | 44 mM Tris, 14.5 mM taurine, 0.1 mM EDTA, pH 9.0 |

### EXAMPLE 1

Bacterial artificial chromosome (BAC) clones identified using physical map information for human chromosome 5q31-q33 publicly available on the Lawrence Berkley National Laboratory Genome Centre web site (LBNL; www-hgc.lbl.gov/biology/bacmap/2.gif) obtained as BAC clone numbers h164 (22f14), c5 (50g20), h187 (35k5), h167 (8e5) and h177 (32d16) from Research Genetics (Huntsville, Ala., USA), and a P1 artificial chromosome (PAC) isolated by PCR using primers with SEQ ID NOS:65 to 68 for the STS markers bac51007T (5′ end of BAC 50g20) and bac51330T (3′ end of BAC 22f14) available on the LBNL website (www hgc.lbl.gov/sts.html) by Genome Systems Inc. (St. Louis, Mo., USA), the BACs and PAC together covering a sub-region of human chromosonal region 5q31-5q33, are sequenced using conventional techniques for an ABI 377 sequence (http://www.pebio.com/ab/about/dna/377/). The resulting genomic DNA sequence is analysed using GENSCAN (Burge and Karlin, J. Mol. Biol. 268:78-94) and GENEMARK version 2.4 (Borodovsky and McIninch, Comp. Chem. 17:123-133) gene-finding programs and BLAST (Altschul et al., J. Mol. Biol. 215:403-410) homology searches against public protein, EST and DNA databases (SWISSPROT, SWISSPROTPLUS, GenBank, Genbank updates, EMBL, GENEMBLPLUS, GenBank EST, EMBL EST, GenBank STS, EMBL STS), the results of which are parsed into a human chromosome 5-specific version of ACeDb (AC. elegans Database; http://www.sanger.ac.uk/Software/Acedb/) for graphic display. From this graphic display significant regions (i.e. genes) are identified by predicted exons and aligned EST/protein hits. A gene AAG6 is initially identified on the graphic display as a GENSCAN-predicted gene covering 20 kb of genomic DNA and comprising 8 exons ranging in size from 69-2816 bp:

| | Nucleotide position in | |
|---|---|---|
| GENSCAN-Predicted Exon | SEQ ID NO:5 | Exon Size (bp) |
| 1 | 1001-1084 | 84 |
| 2 | 4313-4534 | 222 |
| 3 | 8446-8551 | 106 |
| 4 | 8622-11437 | 2816 |
| 5 | 14819-14916 | 98 |
| 6 | 16039-16107 | 69 |
| 7 | 16826-16977 | 152 |
| 8 | 20604-21028 | 425 |

The DNA sequences in GENSCAN-predicted exons 4, 5, 7 and 8 encode a protein having homologies to cadherin-type molecules in a range of organisms, including humans, which suggest that it is a member of the cadherin protein family. Whereas most of the protein homologies are around 40%, a homology of 80% is detected with mouse vascular endothelial cadherin 2 (MVEC2; Telo et al., J. Biol. Chem. 273:17565-17572) indicating that AAG6 may be the human equivalent of MVEC2. In addition, an 83% homology to the MVEC2 mRNA sequence is also detected. BLAST searches against the TIGR database (www.TIGR.ORG) using a 425 bp DNA sequence corresponding to GENSCAN-predicted exon 8 from AAG6 identified on the graphic display reveals a 478 bp EST (TIGR accession no: THC 204848) with 98% sequence identity to the predicted exon. A plasmid clone containing a ˜3.2 kilobase cDNA insert is isolated from a human lung cDNA library of Origene Technologies Inc. (Rockville, Md., USA) by PCR using primers designed using THC 20848 and having SEQ ID NOS: 64, 65 and 66.

The cDNA insert is sequenced using primer-directed walking. The resulting 3.09 kb of insert sequence (SEQ ID NO:1) is analysed using the EditSeq module of Lasergene software (DNASTAR, Inc., Madison, Wis., USA). The cDNA sequence maps to BAC DNA sequence corresponding to GENSCAN-predicted exons 4, 5, 7 and 8. Further EditSeq analysis reveals an open reading frame (ORF) of 2436 nucleotides coding for a protein of 811 amino acids in length (SEQ ID NO:2). Alignment of the AAG6 and MVEC2 protein sequences using the MegAlign module of Lasergene software reveals that both proteins are highly homologous but that 377 amino acids encoded by nucleotides 299-1427 of the MVEC2 cDNA (Telo et al., J. Biol. Chem. 273:17565-175) are absent from the translated AAG6 ORF, indicating that the AAG6 cDNA sequence isolated from the Origene library is incomplete. BLAST analysis using nucleotides 1-1427 of the MVEC2 cDNA sequence indicates that a 916 nucleotide region of genomic DNA (8550 to 9465 in SEQ ID NO:5) 5′ of the AAG6 cDNA sequence may code for the missing amino acids in the AAG6 protein sequence. Therefore, this DNA sequence is appended to the AAG6 cDNA sequence and the new sequence of 4.006 kb (SEQ ID NO:3) is analysed using EditSeq. An ORF of 3564 nucleotides is revealed (from nucleotide position 9-3572 in SEQ ID NO:3) which codes for a protein of 1187 amino acids in size (SEQ ID NO:4). Alignment of this protein sequence with the MVEC2 protein sequence using MegAlign reveals that the amino acids missing in the translation of the ORF of the initial 3.09 kb cDNA sequence are now present.

Analysis of the AAG6 protein sequence using the Protean module of the Lasergene software as well as comparison with the MVEC2 protein structure shows the presence of an extracellular region that can be divided into 6 domains. Five peptide motifs ([L,I,V]X[L,I,V]XDXND[N,H]XP) (where X is any amino acid) found in classical cadherin molecules are found in the extracellular domain. In addition, the protein contains a transmembrane domain and a large cytoplasmic region. The high homology with the MVEC2 protein indicates that AAG6 is the human equivalent of this protein and may display the same functional properties as MVEC2.

Using a 404 bp PCR fragment generated from human genomic DNA using primers having SEQ ID NOS:64 and 65, a northern blot of mRNA from a number of human tissues (human 12-lane MTN blot; Clontech Laboratories UK Ltd., Basingstoke, Hampshire, UK) is probed to examine the expression pattern of AAG6. A single hybridising band of ˜4.5 kb is detected in heart, spleen, placenta, kidney, skeletal muscle, liver and lung. Very faint or no hybridisation is detected for small intestine, brain, colon, thymus and peripheral blood lymphocytes. PCR analysis of first-strand cDNAs derived from 24 different tissues (Rapid-Scan™ Gene Expression Panel; OriGene Technologies Inc., Rockville, Md., USA) using primers having SEQ ID NOS:62 and 63 confirms the Northern blot results. Probing a northern blot of HUVEC and NHBE mRNA with the 404 bp probe shows that the AAG6 gene is expressed in endothelial cells but not in epithelial cells. The expression pattern observed for the AAG6 gene parallels that observed for MVEC2 (Telo et al., J. Biol. Chem. 273:17565-17572).

The size of the hybridising mRNA band detected on northern blots (˜4.5 kb) indicates that SEQ ID NO:3 (4.006 kb) may still not represent the entire messenger RNA from AAG6 and that further non-translated exonic sequence might exist, possibly in the chromosomal DNA corresponding to GENSCAN-predicted exons 1-3 which are not encompassed by SEQ ID NO:3 (SEQ ID NO:3 contains DNA sequences corresponding to GENSCAN-predicted exons 4,5,7 and 8).

### EXAMPLE 2

In this example conformation sensitive gel electrophoresis (CSGE: Ganguly et al., Proc. Natl. Acad. Sci. USA 90:10325-10329; Ganguly and Williams, Hum. Mut. 9:339-343) is used to detect potential sequence changes in PCR-amplified DNA fragments from blood DNA isolated from asthmatic patients. Single base mismatches in DNA heteroduplexes are detected by polyacrylamide gel electrophoresis in the presence of mildly denaturing solvents which amplify the tendency of mismatches to produce conformational changes and result in differential migration of homo-duplexes and heteroduplexes. To generate heteroduplexes, amplified PCR products are thermally denatured, annealed, then analysed by polyacrylamide gel electrophoresis. DNA fragments are visualised by ethidium bromide staining. DNA fragments showing differential electrophoretic migration patterns are then sequenced to confirm the presence of a change to the polynucleotide sequence and the exact nature of this change.

SEQ ID NO:3 is aligned with SEQ ID NO: 5 using the Align module of the FASTA package (Pearson and Lipman, Proc. Natl. Acad. Sci. USA 85:2444-2448) and four exons are identified:

| | Nucleotide position | Nucleotide position | |
|---|---|---|---|
| Exon | in SEQ ID NO:3 | in SEQ ID NO:5 | Exon Size (bp) |
| 1 | 1-2888 | 8550-11437 | 2888 |
| 2 | 2889-2987 | 14819-14917 | 99 |
| 3 | 2988-3140 | 16827-16979 | 153 |
| 4 | 3141-3984 | 20606-21440 | 835 |

PCR primer sets covering the exons (including the exon-intron boundaries), the first 1 kb of intron 1 and 1 kb of the 3′-end of the AAG6 gene are designed using SEQ ID NO:5 and Primer Express™ (version 1.0; Perkin Elmer, P/N 604313). These primer sets (SEQ ID NOS: 6-61) are:

| Primer Set | Forward Primer | Reverse primer |
|---|---|---|
| Exon 1.1 | GCCGGCAGCTCTGGG | CTGAGCAAGCCTTCCTCAGAGT |
| Exon 1.2 | CACTCTCACGGTGAAATACCAAGT | GGATCCGGGTTCGCAGA |
| Exon 1.3 | CCTTTGATGTGCTTGCCACA | TTGGAGTCCAAGACGTTGACCT |
| Exon 1.4 | TGTCATTGTGGGCCCTGAT | TCGACGCAGAATGACCTGG |
| Exon 1.5 | CGCTTCTCATAAAACTGACCGC | TTGTGTCCTCAATCCAAGTCATCT |
| Exon 1.6 | CAGCCCATTGCAAAGTTCTCA | GGTGCATTGTCGTTGATGTCAC |
| Exon 1.7 | CAGGACACAATGGTTTGGTCC | CAATAGCTACTAAGTGAGCAACTGGG |
| Exon 1.8 | TGACATCAACGACAATGCACC | ACGGAGAGGCTGGCTTTTC |
| Exon 1.9 | GCTCACTTAGTAGCTATTGACTCCAACA | TGCCCCCGAGTCTGCA |
| Exon 1.10 | CTCTCCGTGCTTGTGAATGC | CTGAGTCCCTCAGGTGGTCC |
| Exon 1.11 | TTCATCCTCAACCCTCATACGG | GCACGAGGTGGATGTCTGC |
| Exon 1.12 | TGTACTGTTGGGCATCTTCGG | GAGGTTGACCGTGTCTTGCAG |
| Exon 1.13 | GGGCAGTCCCACAAAGATGT | TGAGATGTCGCTGCCGTCT |
| Exon 1.14 | GAGAACCTGAACCTTCCCGAG | GAGGACTGTGCAGGTTATGGG |
| Intron 1.1 | GTCCGGCTGTCTGTGGCT | GGCACCATAAATGCCTTATAGGA |
| Intron 1.2 | CTTAGCTGTGTGATCCTAGGCAAAT | TGAGTACTGACTACATGTCAGATGGACA |
| Intron 1.3 | ACTTCTTCCTAGCTCTGAACCACAG | AACTCCAATCTGCCTAACTGCTAAG |
| Intron 1.4 | ATGAAGAAGGCCAGCATAGGC | GATATGGGTGTGAGATCAGGCA |
| Exon 2 | ACCATCCCCCATCTGATCAC | GGATGCTGTCTGCTGCACC |
| Exon 3 | CCAGGCTGTGTATCCAGTTCC | CCTCCACAGAAACAGAATGGC |
| Exon 4.1 | CCCTTGCCCTGCTCATTG | CCTCCCGCAGACCGAGA |
| Exon 4.2 | CTGAGCCCAACAGGCACG | AGTCACCCTAAAGTTCTCAGGCC |
| Exon 4.3 | CCCAGGTGGAAAGACGGG | TCCTGCCTGTGATACTCCGTG |
| Exon 4.4 | AAGAGCCCCAGGACTAACAGC | CTGTCCAGGCCCAGTGTGA |
| 3′ 1.1 | GGGTGCCAGGAAATGCTCT | GGGTTAATAATACCTCCCTCCCA |
| 3′ 1.2 | ATGTTACCAACTAATCTGCTTCTCAGC | TCCTTTCAACAGTGTTTCCCG |
| 3′ 1.3 | CTGGGCCAACACCAAAACC | GAAGGCTGGCTGCTGTCAGT |
| 3′ 1.4 | AAGTAGCTCTTCAAGTGCGGATG | TATGATGCACATTATCCCTAGTTGG |

Using the above primer sets, 28 polynucleotides are amplified from blood DNA samples from 16 asthmatic patients. PCR reactions are carried out in a reaction volume of 10 μl containing 1×GeneAmpe 10×PCR buffer (Perkin Elmer P/N N808-0240), 13 ng of template DNA, 400 μM of each dNTP (Amersham Life Science Nucleix Plus ™ 25 mM dNTP mix; Prod. No. US77119), 30 ng of each primer, 2 mM MgCl₂ and 0.5 u of AmpliTaq Gold™ polymerase (Perkin-Elmer P/N N808-0242).

Typical thermal cycling conditions using a Biometra UNO II cycler (Part No. 050-603; Anachem Ltd., Luton, UK) are as follows, the sequence Step 2-Step 3-Step 4 being repeated 36 times:

| | | |
|---|---|---|
| Step 1 | 95° C. | 10 min |
| Step 2 | 92° C. | 1 min |
| Step 3 | 60° C. | 1 min |
| Step 4 | 72° C. | 2 min |
| Step 5 | 72° C. | 10 min |

To generate heteroduplexes, 2 μl of PCR product is denatured at 95° C. for 10 minutes and annealed at 68° C. for 30 minutes using a thermal cycler (eg. Biometra UNOII). 2 μl of 2×loading buffer (20% ethylene glycol, 30% formamide, 0.025% xylene cyanol, 0.025% bromphenol blue) is added to each sample before gel analysis.

A standard DNA sequencing apparatus (Owl Scientific S3S; Autogen Bioclear UK Ltd.) is used with a 60 sample comb (Owl Scientific S2S-60A; Autogen Bioclear UK Ltd.) and standard power supply (Biorad, Cat No. 165-5057) equipped with a temperature probe (Biorad, Cat No. 165-5058). A 0.4 mm thick 15% polyacrylamide gel is prepared using a 99:1 ratio of acrylamide to BAP cross linker, 10% ethylene glycol and 15% formamide in 0.5 ×TTE. Gels are pre-run for one hour at 30 watts, limiting the temperature to a maximum of 25ûC (using an electric fan to keep the temperature down if necessary eg. Jencons, Cat No. 292-004). After the pre-run, the wells are flushed with a pipette and the samples are loaded into the wells. The gel is then electrophoresed at 12 watts overnight (15 hours) at 25° C. Fragments greater than 350 bp remain on the gel.

After electrophoresis, the gel plates are separated. The gel is stained by placing the gel in 0.5×TTE containing 1 μg/ml ethidium bromide (Biorad, Cat No. 161-0433) for 10 minutes, followed by destaining in 0.5×TTE for 10 minutes. The gel is then photographed on a UV transilluminator (eg. UVP GDS 7500).

Potential polynucleotide changes are detected by CSGE in one or more of the 16 patients for 18 0f the 28 PCR fragments. For each of these potential changes, the PCR fragment from all 16 patients is subjected to double stranded DNA sequencing on an ABI377 automated sequencer using standard methods (http://www.pebio.com/ab/about/dna/377/) and the resulting DNA sequence is analysed using CONSED software (Gordon et al., Genome Res. 8:195-202) to confirm the presence of a sequence change and to identify the exact base change. All of the 18 potential changes detected by CSGE are confirmed. These changes are shown in the table below, in which #patients indicates the number of patients exhibiting the polymorphism:

| Exon | Alias | Polymorphism | # patients |
|---|---|---|---|
| 385 | Exon 1.1 | C to T | 8 |
| 386 | Exon 1.2 | A to G | 14 |
| 390 | Exon 1.6 | A to C | 12 |
| 393 | Exon 1.9 | G to C | 1 |
| 394 | Exon 1.10 | G to A | 6 |
| 395 | Exon 1.11 | T to C | 12 |
| | Exon 1.11 | C to T | 10 |
| 399 | Intron 1.1 | A to G | 15 |
| 400 | Intron 1.2 | C to T | 5 |
| 401 | Intron 1.3 | C to T | 1 |
| 402 | Intron 1.4 | C to T | 2 |
| | Intron 1.4 | C to T | 3 |
| | Intron 1.4 | 1 bp deletion | 11 |
| 403 | Exon 2 | G to A | 3 |
| 406 | Exon 4.2 | 9 bp insertion | 16 |
| 408 | Exon 4.4 | G to A | 15 |
| 411 | 3′.3 | T to C | 1 |
| 412 | 3′.4 | G to C | 1 |

Sixteen of the detected polynucleotide changes are single nucleotide polymorphisms (SNPs), one is a deletion and the remaining change is a 9bp insertion EXAMPLE 3

This Example relates to the expression of full length AAG6 with a 6 histidine tag at the C-terminus using the Baculovirus system in T.ni Hi5 cells, and to the purification of the resulting polypeptide. A recombinant fragment spanning the extracellular domain of AAG6 is generated as described for MVEC2 (Telo et al., J. Biol. Chem. 273:17565-17572).

1. Construction of a Recombinant AAG6 Baculovirus

A unique EcoRI site is incorporated 5′ to the AAG6 start codon by PCR amplification using the following primer:

5ST

5′-GAAGATCTTCGGAATTCCATCATGATGCAACTTCTGCAACTTCTG-3′

Another primer is used to introduce 6 histidine residues immediately prior to the AAG6 stop codon. This primer also incorporates a unique KpnI site 3′ to the AAG6 stop codon.

3ST

5′-

AAGATCTTCGGTACCTCAATGGTGATGGTGATGGTGCAGGCACCTGCTGCTGCTG-3′

The recombinant “His tagged” version of AAG6 is ligated as a 2467 bp EcoRI/KpnI fragment into EcoRI/Kpnl digested pFastbac1 baculovirus transfer vector (Life Sciences). The recombinant AAG6 sequence is transposed into Bacmid DNA carried by DH10Bac cells (Life Sciences; Bac to Bac Baculovirus expression system). AAG6 recombinant Bacmids are isolated from DH10Bac cells and transfected into Sf9 cells using published protocols (Bac to Bac baculovirus expression system manual; Life Sciences).

2. Amplification of recombinant Baculovirus stocks

The recombinant baculovirus is amplified by infecting Sf9 cells (maintained in SF900 SFMII medium; Life Sciences) at a cell density of 0.5×10⁶ cells/ml and a multiplicity of infection (moi) of 0.01 for 96 hours. Sf9 cells are then centrifuged at 1000×g for 5 minutes. The supernatants containing high titre virus are stored at 4° C.

3. Expression of recombinant AAG6 in Hi5 Cells

Hi5 cells (Invitrogen), maintained at densities of between 3×10⁵ and 3×10⁶ cells/ml in Excell 401 medium (JRH Biosciences; distributed by AMS Biotechnology in either shaker flasks (rotated at 90 RPM) or spinner flasks (stirring at 75 RPM) are infected with the amplified recombinant Baculovirus at a cell density of 2.0×10⁶ at an moi of 2.0 for 60 hours. Following infection Hi5 cells are centrifuged at 1000×g for 5 minutes, the supernatants poured off and the cell pellets frozen at −80° C.

4. Crude lysate preparation

The cells (1×10⁹) are resuspended in 100 ml lysis buffer (20 mM Hepes pH 7.5, 100 mM NaCl, 5% glycerol, 2 mM E-mercaptoethanol, 0.5 mM imidazole, 0.1% Nonidet P-40, 40 μg/ml AEBSF, 0.5 μg/ml leupeptin, 1 μg/ml aprotinin and 0.7 μg/ml pepstatin A). Cells are incubated on ice for 15 min then centrifuged at 39,000×g for 30 min at 4° C. The sample is filtered through a 0.22 μm filter immediately prior to use.

5. Metal chelate affinity chromatography

Metal chelate affinity chromatography is carried out at room temperature with a column attached to a BioCAD chromatography workstation. A 20 ml Poros MC/M (16 mmD×100 mmL) column is charged with Ni²⁺ prior to use and after each injection. To charge with Ni²⁺, the column is washed with 10 column volumes (CV) 50 mM EDTA pH 8, 1 M NaCl followed by 10CV water. The column is charged with 500 ml 0.1 M NiSO4 pH 4.5-5, washed with 10CV water, then any unbound Ni²⁺removed by washing with 5CV 0.3 M NaCl. All steps are performed with a flow rate of 20 ml/min. The charged MC/M column is equilibrated with 5CV Buffer B (20 mM Hepes pH 7.5, 500 mM NaCl, 5% glycerol, 2 mM β-mercaptoethanol, 1 mM PMSF, 5 mM imidazole) to saturate the sites followed by 10CV Buffer A (as Buffer B except 0.5 mM imidazole). 90-95 ml of the crude lysate is loaded onto the column per run at a flow rate of 20 ml/min. Subsequent steps are carried out with a flow rate of 30 ml/min. Any unbound material is removed by washing with 12 CV buffer A and AAG6 eluted by applying a 0-50% Buffer B gradient over 10 CV. Fractions (8 ml) are collected over the gradient. AAG6-containing fractions are combined and protease inhibitors added to the final concentrations described for the lysis buffer above. DTT is also added to a final concentration of 1 mM. The combined fractions are dialysed overnight against 4 liters 20 mM Tris-HCl pH 7.5, 1 mM DTT, 0.2 mM PMSF at 4° C.

6. Ion Echange (Anion Exchange) Chromatography

Resource™ Q chromatography is carried out at 4° C. with a column attached to an FPLC workstation (Amersham Pharmacia Biotech). A 6 ml Resource™ Q column (16 mmD×30 mmL) is equilibrated with 10 CV Buffer C (20 mM Tris-HCl pH 7.5, 1 mM DTT) at a flow rate of 2 ml/min. The dialysed metal chelate eluate is applied to the column and washed with 10 CV Buffer C. The protein is eluted by applying a 0-100% Buffer D gradient (20 mM Tris-HCl pH 7.5, 1 mM DTT, 1 M NaCl) over 10 CV. Fractions (3 ml) are collected on eluting the column.

7. Gel Filtration

Gel Filtration chromatography is carried out at 4° C. with a column attached to a BioCAD SPRINT chromatography workstation (PE Biosystems). A 24 ml (10 mmD×300 mmL) Superdex 200 HR (Amersham Pharmacia Biotech) column is equilibrated with 10 CV Buffer E (20 mM Tris-HCl pH7.5, 1 mM DTT, 150 mM NaCl) at a flow rate of 0.5 ml/min. The ion exchange eluate is applied to the column and eluted with Buffer E. Fractions ( 1 ml) throughout the purification run are collected and analysed.

8. Sample Concentration

Samples are concentrated approximately 10-fold using a Millipore Ultrafree-15 centrifugation device (MW cut-off 50 kDa) at 4° C. The device is pre-rinsed with water prior to use. The final storage buffer used for long term storage at−80° C. is 20 mM Hepes pH 7.5, 1 mM DTT, 100 mM NaCl, 5% glycerol. Glycerol can be omitted from the sample for storage at 4° C.

### EXAMPLE 4

This example relates to the production of polyclonal antibodies against AAG6 protein purified as described in Example 3. Polyclonal antibodies against a recombinant fragment spanning the extracellular domain are generated as described by Telo et al. (J. Biol. Chem. 273:17565-17572).

### Immunisation of Rabbits

Dutch rabbits (Harlen-Olac) are immunised at 4 subcutaneous sites with 500 μg purified AAG6 protein in PBS according to the following schedule:

| DAYS | IMMUNISATIONS |
|---|---|
| 0 | 1^{st} immunisation 1:1 in complete Freund's adjuvant |
| 15 | 1^{st} boost 1:1 in incomplete Freund's adjuvant |
| 45 | 2^{nd} boost 1:1 in incomplete Freund's adjuvant |
| 55 | 1^{st} test bleed from the ear artery |
| Every month | Boost 1:1 in incomplete Freund's adjuvant until a good |
| | antibody response is obtained |

Test bleeds (500 μl) are taken and the serum assessed for antibody titre. Serum is collected when a maximum titre is reached. This is done by collecting blood (10 ml) and allowing it to clot for 2 hours at 4° C. The blood is centrifuged at 1000×g for 5 minutes to separate the serum. The serum is removed and stored at −20° C. until assayed.

### ELISA Screening

Nunc-Immuno Plate Maxisorp 96 well plates (Nunc, Fisher Scientific UK, Loughborough, UK) are used as a solid support and coated with the purified AAG6 protein (100 ng/well) overnight at 4° C. The plates are blocked for 3 hours at 37° C. with PBS containing 2% BSA (Sigma) and 0.02% NaN₃ (Sigma). After blocking, plates are incubated overnight at room temperature with serum in different dilutions of PBS. The presence of polyclonal antibodies is checked with both biotin labelled IgG-antibodies to rabbit (Goat anti-rabbit IgG antiserum, 1:25000 dilution), with an incubation time of 40 min. Alkaline phosphatase conjugated streptavidin (Immununo Research, Dianova, CH) is then added at a dilution of 1:10000. Development of the reaction is carried out by adding an alkaline phosphatase substrate (Sigma, f.c. 1 mg/ml) dissolved in diethanolamine. After 45 min. absorbance is read at 405 nm with a reference of 490 nm with an ELISA plate reader (Bio-rad laboratories Ltd., Hemel Hempstead, UK).

### Purification

5 ml protein A-agarose is poured into a chromatography column and washed with 6 column volumes of 0.1 M tris (hydroxymethyl) methylamine (Tris) buffer pH 7.5. The rabbit serum containing anti-AAG6 antibodies is diluted (½) with Tris buffer and added to the protein A-agarose. Unbound proteins are removed by washing the column with 6 volumes of Tris buffer. The IgG is eluted off the column with three column volumes of 0.1 M glycine buffer pH 3.0 and collected as 1 ml fractions into tubes containing 28 μl of 1 M Tris. The fractions which are positive for protein content are checked for purity by SDS-PAGE under reducing conditions. Two bands at 50 and 25 Kd are visualised corresponding to the heavy and light chains of an immunoglobulin molecule. Fractions containing only immunoglobulin are pooled, re-checked for protein concentration and stored at −20° C.

### EXAMPLE 5

This example describes the preparation of monoclonal antibodies against AAG6 protein purified as described in Example 3.

### Immunisation of Mice

Female Balb/c mice are immunised intraperitoneally with 100 μg of AAG6 protein in PBS according to the schedule given below:

| DAYS | IMMUNISATIONS |
|---|---|
| 1 | 1^{st} immunisation 1:1 with complete |
| | Freund's adjuvant |
| 14 | 1^{st} boost 1:1 with incomplete Freund's |
| | adjuvant |
| 21 | 2^{nd} boost 1:1 with incomplete Freund's |
| | adjuvant |
| 28-30 | Three final boosts in PBS |
| 31 | Fusion with mouse myeloma cells |

Serum is assessed for antibody titre by ELISA (Example 4) after the animal is sacrificed for the preparation of spleen cells for fusion. If antibody titre is sufficient, (1/1000 to 1/100,000), the hybridomas are screened, otherwise discarded.

### Preparation of Myeloma Cells

Sp2/0 murine myeloma cells (ATCC #CRL 1581; maintained in culture medium containing 20 μg/ml 8-azaguanine) are cultivated for one week before fusion in RPMI 1640 (8-azaguanine is not included), 10% (v/v) FCS and 1% penicillin-streptomycin (50IU/ml and 50 μg/ml, respectively). The cells are harvested by centrifugation (200×g for 5 min) and washed three times in cold RPMI 1640. Approximately 2.5×10⁶ cells are used per 96 well microtitre plate.

### Preparation of Spleen Cell Suspension

The mouse is killed by an overdose of anesthetic (Forene), the spleen dissected and pressed through a cell strainer (70 μm mesh cell strainer; Becton & Dickinson, Oxford, UK, Cat. No 2350). The cell suspension is washed three times in RPMI 1640 (as above) and counted: 5.10⁶ cells/96 well plate are necessary.

### Fusion of Myeloma Cells and Spleen Cells

The spleen and myeloma cells are mixed (2:1), centrifuged (200×g for 5 min) and the pellet warmed in a 37° C. water bath. Prewarmed polyethylene glycol 4000 ( 1 ml per 10⁸ cells) is added slowly over one minute, then 20 ml of prewarmed wash medium over two minutes. After centrifugation the pellet is carefully resuspended in selection medium (RPMI 1640, 10% FCS, 1% penicillin-streptomycin, 10% BM condimed H1 (feeder cell replacement from Boehringer Mannheim, Lewes, UK; Cat. No. 1 088 947), 10% HAT-media supplement (hypoxanthine, aminopterin and thymidine to select against unfused myeloma cells; Boehringer Mannheim, Lewes, UK; Cat. No. 644 579) and plated, 200 μl/well of a 96 well microtitre plate.

After five days clusters of hybrid cells can be identified by examining the bottom of the microtitre wells with an inverted microscope. After 10-14 days the culture supernatant is tested for the presence of antibodies by ELISA (example 4). The positive clones are expanded in a 24 well assay plate and retested.

### Cloning of Positive Hybridomas

The expanded clones which are still positive are cloned by limiting dilution. Cells are diluted serially in four dilutions steps in a 96 well microtitre plate; 5, 2, 1 and 0.5 cells/well. HAT-media supplement is replaced with HT-media supplement (Boehringer Mannheim, Lewes, UK; Cat. No. 623 091). After approximately one week the cells are screened by ELISA (Example 4). The cells of those wells containing a single positive clone are expanded.

### Production of Monoclonal Antibody Supernatant

The cells are grown in culture flasks in standard medium (RPMI 1640, 10% (v/v) FCS and 1% penicillin-streptomycin) until the hybridomas overgrow and die. The debris is removed by centrifugation and the supernatant containing the antibodies is titred using ELISA (Example 4) before storing under sterile conditions at 4° C., −20° C. or −70° C. .

1. An isolated polynucleotide comprising a nucleotide sequence encoding a polypeptide comprising the amino acid sequence of SEQ ID NO:2 or SEQ ID NO:4 or a functionally equivalent variant of said amino acid sequence.

2. An isolated polynucleotide according to claim 1 which is cDNA comprising the nucleotide sequence of SEQ ID NO:1 or SEQ ID NO:3 or a DNA comprising a nucleotide sequence which hybridises to SEQ ID NO:1 or SEQ ID NO:3 under stringent conditions.

3. An isolated polynucleotide according to claim 1 which is a genomic DNA comprising the nucleotide sequence of SEQ ID NO:5 or a DNA comprising a nucleotide sequence which hybridises to SEQ ID NO:5 under stringent conditions.

4. An isolated polynucleotide comprising a consecutive 20 base pair nucleotide portion identical in sequence to a consecutive 20 base pair portion of SEQ ID NO:1 or SEQ ID NO:3 or SEQ ID NO:5.

5. An isolated polypeptide comprising the amino acid sequence of SEQ ID NO:2 or SEQ ID NO:4 or a functionally equivalent variant thereof.

6. An isolated polypeptide comprising a consecutive 10 amino acid portion identical in sequence to a consecutive 10 amino acid portion of SEQ ID NO:2 or SEQ ID NO:4.

7. A method of producing a polypeptide according to claim 5 which comprises culturing a host cell containing an expression vector containing a polynucleotide sequence as specified in claim 1, under conditions suitable for expression of the polypeptide and recovering the polypeptide from the host cell culture.

8. An expression vector containing a polynucleotide sequence as specified in claim 1.

9. An antibody which is immunoreactive with a polypeptide according to claim 5.

10. An antisense oligonucleotide comprising a nucleotide sequence complementary to that of a polynucleotide encoding a polypeptide according to claim 5 or a variant thereof having a polymorphism correlated with a disease.

11. A polynucleotide probe comprising at least 15 contiguous nucleotides of a polynucleotide according to claim 1, or a complement thereof.

12. A pharmaceutical composition comprising a polynucleotide according to claim 1, a polypeptide comprising the amino acid sequence of SEQ ID NO:2 or SEQ ID NO:4 or a functionally equivalent variant thereof, an antibody which is immunoreactive with said polypeptide or a variant thereof having a polymorphism correlated with a disease, or an antisense oligonucleotide comprising a nucleotide sequence complementary to that of said polynucleotide or a variant thereof having a polymorphism correlated with a disease, optionally together with a pharmaceutically acceptable carrier.

13. A method of treating an inflammatory or obstructive airways disease which comprises administering to a subject in need thereof an effective amount of a polynucleotide according to claim 1, a polypeptide comprising the amino acid sequence of SEQ ID NO:2 or SEQ ID NO:4 or a functionally equivalent variant thereof, an antibody which is immunoreactive with said polypeptide or a variant thereof having a polymorphism correlated with a disease, or an antisense oligonucleotide comprising a nucleotide sequence complementary to that of said polynucleotide or a variant thereof having a polymorphism correlated with a disease.

14. A method of detecting genetic abnormality in a subject which comprises incubating a genetic sample from the subject with a polynucleotide probe according to claim 11, under conditions where the probe hybridises to complementary polynucleotide sequence, to produce a first reaction product, and comparing the first reaction product to a control reaction product obtained with a normal genetic sample, where a difference between the first reaction product and the control reaction product indicates a genetic abnormality in the subject or a predisposition to developing a disease.

15. A method of detecting the presence of a polynucleotide according to claim 1 in a cell or tissue which comprises contacting DNA from the cell or tissue with a polynucleotide probe comprising at least 15 contiguous nucleotides of a polynucleotide according to claim 1 under conditions where the probe is specifically hybridizable with a polynucleotide according to claim 1, and detecting whether hybridization occurs.

16. A method of detecting an abnormality in the nucleotide sequence of a polynucleotide according to claim 1 in a patient which comprises amplifying a target nucleotide sequence, in DNA isolated from the patient, by a polymerase chain reaction using a pair of primers which target the sequence to be amplified and analysing the amplified sequence to determine any polymorphism present therein.

17. A pair of oligonucleotides useful as primers for amplification of a fragment of a polynucleotide according to claim 1, each oligonucleotide of said pair being at least 15 nucleotides in length and said pair having sequences such that when used in a polymerase chain reaction with human genomic DNA or a suitable human cDNA target, they result in synthesis of a DNA fragment containing part or all of the nucleotide sequence of a polynucleotide according to claim 1.

18. A variant of a polynucleotide comprising a nucleotide sequence encoding a polypeptide comprising amino acid sequence SEQ ID NO:2 or SEQ ID NO:4, which variant contains a sequence polymorphism correlated with asthma.

19. A method of determining predisposition of a patient to asthma which comprises identifying in DNA from the patient a sequence polymorphism or haplotype in a nucleotide sequence encoding a polypeptide comprising amino acid sequence SEQ ID NO:2 or SEQ ID NO:4 as compared with a normal control DNA from a non-asthmatic subject, which correlates with asthma.

20. A method of identifying a substance which modulates the activity of a polypeptide comprising the amino acid sequence of SEQ ID NO:2 or SEQ ID NO:4 or a functionally equivalent variant thereof, or a polypeptide encoded by a variant of a polynucleotide comprising a nucleotide sequence encoding a polypeptide comprising amino acid sequence SEQ ID NO:2 or SEQ ID NO:4, which variant contains a sequence polymorphism, comprising combining a candidate substance with said polypeptide and measuring the effect of the candidate substance on said activity.

21. A method for pharmacogenomically selecting a therapy to administer to an individual having asthma, comprising determining an AAG6 genetic profile of an individual and comparing the individual's AAG6 genetic profile to an AAG6 genetic population profile, to thereby select a therapy for administration to the individual.

22. A method according to claim 21, wherein determining the AAG6 genetic profile of an individual comprises determining the identity of a single nucleotide polymorphism.


SEQUENCE LISTING (70 sequences)

SEQ ID NO: 1 - DNA, 3090 bp, Homo sapiens
ggtcattctgcgtcgacctctagactatgaaaagaaccctgcctacgaggtggatgttcaggcaagggacctgggtcccaatcctatcccagcccattgcaaagttctcatcaaggttctggatgtcaatgacaacatcccaagcatccacgtcacatgggcctcccagccatcactggtgtcagaagctcttcccaaggacagttttattgctcttgtcatggcagatgacttggattcaggaaacaatggtttggtccactgctggctgagccaagagctgggccacttcaggctgaaaagaactaatggcaacacatacatgttgctaaccaatgccacactggacagagagcagtggcccaaatataccctcactctgttagcccaagaccaaggactccagcccttatcagccaagaaacagctcagcattcagatcagtgacatcaacgacaatgcacctgtgtttgagaaaagcaggtatgaagtctccacgcgggaaaacaacttaccctctcttcacctcattaccatcaaggctcatgatgcagacttgggcattaatggaaaagtctcataccgcatccaggactccccagttgctcacttagtagctattgactccaacacaggagaggtcactgctcagaggtcactgaactatgaagagatggccggctttgagttccaggtgatcgcagaggacagcgggcaacccatgcttgcatccagtgtctctgtgtgggtcagcctcttggatgccaatgataatgccccagaggtggtccagcctgtgctcagcgatggaaaagccagcctctccgtgcttgtgaatgcctccacaggccacctgctggtgcccatcgagactcccaatggcttgggcccagcgggcactgacacacctccactggccactcacagctcccggccattccttttgacaaccattgtggcaagagatgcagactcgggggcaaatggagagcccctctacagcatccgcagtggaaatgaagcccacctcttcatcctcaaccctcatacggggcagctgttcgtcaatgtcaccaatgccagcagcctcattgggagtgagtgggagctggagatagtagtagaggaccagggaagcccccccttacagacccgagccctgttgagggtcatgtttgtcaccagtgtggaccacctgagggactcagcccgcaagcctggggctttgagcatgtcgatgctgacggtgatctgcctggctgtactgctgggcatcttcgggttgatcctggctttgttcatgtccatctgccggacagaaaagaaggacaacagggcctacaactgtcgggaggccgagtccacctaccgccagcagcccaagaggccccagaaacacattcagaaggcagacatccacctcgtgcctgtgctcaggggtcaggcaggtgagccttgtgaagtcgggcagtcccacaaagatgtggacaaggaggcgatgatggaagcaggctgggacccctgcctgcaggcccccttccacctcaccccgaccctgtacaggacgctgcgtaatcaaggcaaccaaggagcaccggcggagagccgagaggtgctgcaagacacggtcaacctccttttcaaccatcccaggcagaggaatgcctcccgggagaacctgaaccttcccgagccccagcctgccacaggccagccacgttccaggcctctgaaggttgcaggcagccccacagggaggctggctggagaccagggcagtgaggaagccccacagaggccaccagcctcctctgcaaccctgagacggcagcgacatctcaatggcaaagtgtcccctgagaaagaatcagggccccgtcagatcctgcggagcctggtccggctgtctgtggctgccttcgccgagcggaaccccgtggaggagctcactgtggattctcctcctgttcagcaaatctcccagctgctgtccttgctgcatcagggccaattccagcccaaaccaaaccaccgaggaaataagtacttggccaagccaggaggcagcaggagtgcaatcccagacacagatggcccaagtgcaagggctggaggccagacagacccagaacaggaggaagggcctttggatcctgaagaggacctctctgtgaagcaactgctagaagaagagctgtcaagtctgctggaccccagcacaggtctggccctggaccggctgagcgcccctgacccggcctggatggcgagactctctttgcccctcaccaccaactaccgtgacaatgtgatctccccggatgctgcagccacggaggagccgaggaccttccagacgttcggcaaggcagaggcaccagagctgagcccaacaggcacgaggctggccagcacctttgtctcggagatgagctcactgctggagatgctgctggaacagcgctccagcatgcccgtggaggccgcctccgaggcgctgcggcggctctcggtctgcgggaggaccctcagtttagacttggccaccagtgcagcctcaggcatgaaagtgcaaggggacccaggtggaaagacggggactgagggcaagagcagaggcagcagcagcagcagcagcagcagcaggtgcctgtgaacatacctcagacgcctctggatccaagaaccaggggcctgaggatctgtggacaagagctggtttctaaaatcttgtaactcactagctagcggcggcctgagaactttagggtgactgatgctacccccacagaggaggcaagagccccaggactaacagctgactgaccaaagcagccccttgtaagcagctctgagtcttttggaggacagggacggtttgtggctgagataagtgtttcctggcaaaacatatgtggagcacaaagggtcagtcctctggcagaacagatgccacggagtatcacaggcaggaaaggatggccttcttgggtagcaggagtcagggggctgtaccctgggggtgccaggaaatgctctctgacctatcaataaaggaaaagcagtgaaaaaaaaaaaaaaaaaaaaaaa

SEQ ID NO: 2 - protein, 811 aa, Homo sapiens
MADDLDSGNNGLVHCWLSQELGHFRLKRTNGNTYMLLTNATLDREQWPKYTLTLLAQDQGLQPLSAKKQLSIQISDINDNAPVFEKSRYEVSTRENNLPSLHLITIKAHDADLGINGKVSYRIQDSPVAHLVAIDSNTGEVTAQRSLNYEEMAGFEFQVIAEDSGQPMLASSVSVWVSLLDANDNAPEVVQPVLSDGKASLSVLVNASTGHLLVPIETPNGLGPAGTDTPPLATHSSRPFLLTTIVARDADSGANGEPLYSIRSGNEAHLFILNPHTGQLFVNVTNASSLIGSEWELEIVVEDQGSPPLQTRALLRVMFVTSVDHLRDSARKPGALSMSMLTVICLAVLLGIFGLILALFMSICRTEKKDNRAYNCREAESTYRQQPKRPQKHIQKADIHLVPVLRGQAGEPCEVGQSHKDVDKEAMMEAGWDPCLQAPFHLTPTLYRTLRNQGNQGAPAESREVLQDTVNLLFNHPRQRNASRENLNLPEPQPATGQPRSRPLKVAGSPTGRLAGDQGSEEAPQRPPASSATLRRQRHLNGKVSPEKESGPRQILRSLVRLSVAAFAERNPVEELTVDSPPVQQISQLLSLLHQGQFQPKPNHRGNKYLAKPGGSRSAIPDTDGPSARAGGQTDPEQEEGPLDPEEDLSVKQLLEEELSSLLDPSTGLALDRLSAPDPAWMARLSLPLTTNYRDNVISPDAAATEEPRTFQTFGKAEAPELSPTGTRLASTFVSEMSSLLEMLLEQRSSMPVEAASEALRRLSVCGRTLSLDLATSAASGMKVQGDPGGKTGTEGKSRGSSSSSSSSRCL

SEQ ID NO: 3 - DNA, 4006 bp, Homo sapiens
cggtaagcatgatgcaacttctgcaacttctgctggggcttttggggccaggtggctacttatttcttttaggggattgtcaggaggtgaccactctcacggtgaaataccaagtgtcagaggaagtgccatctggtacagtgatcgggaagctgtcccaggaactgggccgggaggagaggcggaggcaagctggggctgccttccaggtgttgcagctgcctcaggcgctccccattcaggtggactctgaggaaggcttgctcagcacaggcaggcggctggatcgagagcagctgtgccgacagtgggatccctgcctggtttcctttgatgtgcttgccacaggggatttggctctgatccatgtggagatccaagtgctggacatcaatgaccaccagccacggtttcccaaaggcgagcaggagctggaaatctctgagagcgcctctctgcgaacccggatccccctggacagagctcttgacccagacacaggccctaacaccctgcacacctacactctgtctcccagtgagcactttgccttggatgtcattgtgggccctgatgagaccaaacatgcagaactcatagtggtgaaggagctggacagggaaatccattcattttttgatctggtgttaactgcctatgacaatgggaacccccccaagtcaggtaccagcttggtcaaggtcaacgtcttggactccaatgacaatagccctgcgtttgctgagagttcactggcactggaaatccaagaagatgctgcacctggtacgcttctcataaaactgaccgccacagaccctgaccaaggccccaatggggaggtggagttcttcctcagtaagcacatgcctccagaggtgctggacaccttcagtattgatgccaagacaggccaggtcattctgcgtcgacctctagactatgaaaagaaccctgcctacgaggtggatgttcaggcaagggacctgggtcccaatcctatcccagcccattgcaaagttctcatcaaggttctggatgtcaatgacaacatcccaagcatccacgtcacatgggcctcccagccatcactggtgtcagaagctcttcccaaggacagttttattgctcttgtcatggcagatgacttggattcaggaaacaatggtttggtccactgctggctgagccaagagctgggccacttcaggctgaaaagaactaatggcaacacatacatgttgctaaccaatgccacactggacagagagcagtggcccaaatataccctcactctgttagcccaagaccaaggactccagcccttatcagccaagaaacagctcagcattcagatcagtgacatcaacgacaatgcacctgtgtttgagaaaagcaggtatgaagtctccacgcgggaaaacaacttaccctctcttcacctcattaccatcaaggctcatgatgcagacttgggcattaatggaaaagtctcataccgcatccaggactccccagttgctcacttagtagctattgactccaacacaggagaggtcactgctcagaggtcactgaactatgaagagatggccggctttgagttccaggtgatcgcagaggacagcgggcaacccatgcttgcatccagtgtctctgtgtgggtcagcctcttggatgccaatgataatgccccagaggtggtccagcctgtgctcagcgatggaaaagccagcctctccgtgcttgtgaatgcctccacaggccacctgctggtgcccatcgagactcccaatggcttgggcccagcgggcactgacacacctccactggccactcacagctcccggccattccttttgacaaccattgtggcaagagatgcagactcgggggcaaatggagagcccctctacagcatccgcagtggaaatgaagcccacctcttcatcctcaaccctcatacggggcagctgttcgtcaatgtcaccaatgccagcagcctcattgggagtgagtgggagctggagatagtagtagaggaccagggaagcccccccttacagacccgagccctgttgagggtcatgtttgtcaccagtgtggaccacctgagggactcagcccgcaagcctggggctttgagcatgtcgatgctgacggtgatctgcctggctgtactgctgggcatcttcgggttgatcctggctttgttcatgtccatctgccggacagaaaagaaggacaacagggcctacaactgtcgggaggccgagtccacctaccgccagcagcccaagaggccccagaaacacattcagaaggcagacatccacctcgtgcctgtgctcaggggtcaggcaggtgagccttgtgaagtcgggcagtcccacaaagatgtggacaaggaggcgatgatggaagcaggctgggacccctgcctgcaggcccccttccacctcaccccgaccctgtacaggacgctgcgtaatcaaggcaaccaaggagcaccggcggagagccgagaggtgctgcaagacacggtcaacctccttttcaaccatcccaggcagaggaatgcctcccgggagaacctgaaccttcccgagccccagcctgccacaggccagccacgttccaggcctctgaaggttgcaggcagccccacagggaggctggctggagaccagggcagtgaggaagccccacagaggccaccagcctcctctgcaaccctgagacggcagcgacatctcaatggcaaagtgtcccctgagaaagaatcagggccccgtcagatcctgcggagcctggtccggctgtctgtggctgccttcgccgagcggaaccccgtggaggagctcactgtggattctcctcctgttcagcaaatctcccagctgctgtccttgctgcatcagggccaattccagcccaaaccaaaccaccgaggaaataagtacttggccaagccaggaggcagcaggagtgcaatcccagacacagatggcccaagtgcaagggctggaggccagacagacccagaacaggaggaagggcctttggatcctgaagaggacctctctgtgaagcaactgctagaagaagagctgtcaagtctgctggaccccagcacaggtctggccctggaccggctgagcgcccctgacccggcctggatggcgagactctctttgcccctcaccaccaactaccgtgacaatgtgatctccccggatgctgcagccacggaggagccgaggaccttccagacgttcggcaaggcagaggcaccagagctgagcccaacaggcacgaggctggccagcacctttgtctcggagatgagctcactgctggagatgctgctggaacagcgctccagcatgcccgtggaggccgcctccgaggcgctgcggcggctctcggtctgcgggaggaccctcagtttagacttggccaccagtgcagcctcaggcatgaaagtgcaaggggacccaggtggaaagacggggactgagggcaagagcagaggcagcagcagcagcagcagcagcagcaggtgcctgtgaacatacctcagacgcctctggatccaagaaccaggggcctgaggatctgtggacaagagctggtttctaaaatcttgtaactcactagctagcggcggcctgagaactttagggtgactgatgctacccccacagaggaggcaagagccccaggactaacagctgactgaccaaagcagccccttgtaagcagctctgagtcttttggaggacagggacggtttgtggctgagataagtgtttcctggcaaaacatatgtggagcacaaagggtcagtcctctggcagaacagatgccacggagtatcacaggcaggaaaggatggccttcttgggtagcaggagtcagggggctgtaccctgggggtgccaggaaatgctctctgacctatcaataaaggaaaagcagtgaaaaaaaaaaaaaaaaaaaaaaa

SEQ ID NO: 4 - protein, 1187 aa, Homo sapiens
MMQLLQLLLGLLGPGGYLFLLGDCQEVTTLTVKYQVSEEVPSGTVIGKLSQELGREERRRQAGAAFQVLQLPQALPIQVDSEEGLLSTGRRLDREQLCRQWDPCLVSFDVLATGDLALIHVEIQVLDINDHQPRFPKGEQELEISESASLRTRIPLDRALDPDTGPNTLHTYTLSPSEHFALDVIVGPDETKHAELIVVKELDREIHSFFDLVLTAYDNGNPPKSGTSLVKVNVLDSNDNSPAFAESSLALEIQEDAAPGTLLIKLTATDPDQGPNGEVEFFLSKHMPPEVLDTFSIDAKTGQVILRRPLDYEKNPAYEVDVQARDLGPNPIPAHCKVLIKVLDVNDNIPSIHVTWASQPSLVSEALPKDSFIALVMADDLDSGNNGLVHCWLSQELGHFRLKRTNGNTYMLLTNATLDREQWPKYTLTLLAQDQGLQPLSAKKQLSIQISDINDNAPVFEKSRYEVSTRENNLPSLHLITIKAHDADLGINGKVSYRIQDSPVAHLVAIDSNTGEVTAQRSLNYEEMAGFEFQVIAEDSGQPMLASSVSVWVSLLDANDNAPEVVQPVLSDGKASLSVLVNASTGHLLVPIETPNGLGPAGTDTPPLATHSSRPFLLTTIVARDADSGANGEPLYSIRSGNEAHLFILNPHTGQLFVNVTNASSLIGSEWELEIVVEDQGSPPLQTRALLRVMFVTSVDHLRDSARKPGALSMSMLTVICLAVLLGIFGLILALFMSICRTEKKDNRAYNCREAESTYRQQPKRPQKHIQKADIHLVPVLRGQAGEPCEVGQSHKDVDKEAMMEAGWDPCLQAPFHLTPTLYRTLRNQGNQGAPAESREVLQDTVNLLFNHPRQRNASRENLNLPEPQPATGQPRSRPLKVAGSPTGRLAGDQGSEEAPQRPPASSATLRRQRHLNGKVSPEKESGPRQILRSLVRLSVAAFAERNPVEELTVDSPPVQQISQLLSLLHQGQFQPKPNHRGNKYLAKPGGSRSAIPDTDGPSARAGGQTDPEQEEGPLDPEEDLSVKQLLEEELSSLLDPSTGLALDRLSAPDPAWMARLSLPLTTNYRDNVISPDAAATEEPRTFQTFGKAEAPELSPTGTRLASTFVSEMSSLLEMLLEQRSSMPVEAASEALRRLSVCGRTLSLDLATSAASGMKVQGDPGGKTGTEGKSRGSSSSSSSSRCL

SEQ ID NO: 5 - DNA, 22494 bp, Homo sapiens
tttacacaattactatttatgacatttttatttatttttaatataaaaagatgtaataaaagacagcaatgtcataaagttgaagtcaggtgtattatactttggggttttgttttgtttttgagaccgagtctcactctgtcacccaggctagagtgcagtggcacaatctcagctcacggcaacctctgcctcccggtagagggttcaagcgattctcgtgcctcagcctcctgagtagctgggattccaggtgcacaccaccacatctggctaatttttgtatttttagtagggatggggtttcaccatgttgcccaggctggtcttgaactcctgggctcaagtgatccacccgctttggcttcccaaagtgccgcgactacagatgtgagccacggcgcctgggccactttttttttttttttttaacttttaagttcatgggtacatgtgcaggtttgttatgtaggtaaactcgtgtcacgggggtttgttgtacagattatttcgtcacccagtgacgaaatattaagtctagtactttttattcttcctgatcctctccctcctctcatcctctcatcctctaccctgtagtaggtaggccccagtgtgtgttgttccccactttgtgaccatgtgttctcatcatttagcttccacttacaagtgagaatatgtggtatttggttttctgttcctgtgctagtttgctaaagatgatggaggcactaaggtttggccagtatcacacagtggtggagccagggttagagccgattcctcatcagtctgactctctccaggagcctgtcagagaataagggttttttgtaacaaattcacagagagtaaaatagttctggccttaagaactaaacgggaaggccctggggaagttaggattcgagtttatgacttaaggataggtgagaatgagaacacagacagagaaagagacccaactcccaccccctgtccccagggacaccggatgatgacggagcctagggagaggagaggttacagtgtaccacctagaccagaggtcgggacccaggccacggagtggagagtagaagtgagtcacaaactatggcttgtcactataggggttagcagacctgacggtgcctgcatttgttcccatccatcactggcatctggtagctgtcccgaaactttgctgaactcatcccaccatcccaggggggactgcctccttttccatggccaccactcgcaagcactcctgcaacaagcccttgaacacagaggggcagcagtgtggagaggaaaaccctgcccaacttttggataaaactgaaccttaatgagccctaccccagtacagaagtgtccctgacttcttgctggtctcttgtcattgtgtatttgctcatcatgtagatcttaaaagtctattatcatgtgggcctctgaaacattgtttttttttaaaaaaaattagaggtttcaggccaggcgtggtggctcatgcctgttatcccagccctttgggaggccgaggcaggtggaccacctgagatcaggagtttgagaccagcctggccaatgtggtgaaaccctgcttctactaaaaatacaaaaaaaaaaaaaaaaaattagccgggcttggtggcgggcacctgtaatcccagctactcgggaggccgagacaggagaatcacttgaacccaagaggcagaggttgcggtgagcccagatcgcaccattgcactccagcctgggcaacagaatgagactctgtctcaaaaaataaaacaaaatttaaaaaaatagaggcttcatcctaaaaaattggaagtgcctacactagaagcacaattataaataacgcttaaagtgctacttatatcccaggccccactctaagtgctgtatgttaactaagctaaacccaacatcaaccctataacatagcaccatcatcatcatcatcattcctattttaaagctaaggaaactgatcatagataattgacttgccatcgtcacacagatggtaaattacagagccaatccctgaacccaggcaattggtgccctgggcccacatcattaaccccattgctatgcttagaccaagtcaatacttggctcaaggaaagtggtcagctgggtctgtaaggagaaatagccagattacctgcgtgaccagcttccctggccagtgcaacctaaacaataactcaggtcaggtcagcccagggttatataaagacagactgctacaagggcttgaggtggctgacagtataaaatgttttctgtgcttttgatgcaagctaaaatttgatttacaataataggcacagaatgatcatgtttatactcgcaacctggcattttattattattcccctgaataacagtgaaaagagttttgtaaccccaaggtaacatggctctgtaaggaggaaagaggggtgagatttagcagcagctgccatcataaatcagtgtgtggctgtttctctcagagctccaggctcaactcctggctgtcataaacttataaagggaagccccatgagaatttatgtaacgatgggaagactcattttccttcctgcaagagcctttctgaagcaaggagagaattaggccgattagattctatatgaagaaaaaaaccaagccaatggtttagaggaagggaaaatgtgggtcaacttgagctacttcccttgccaaaaaaatgaccttgtaaatagagaggaacatgctaaattccaccaaataaatgagtgactggatgactgaatgatttaataaatgagtgtaatctaggcttcctccagggtggttctttcgtgtaaaacagttcgatcttggagtcacccagaaatgctgaacacataacaaggccaatcaacagagttcgcttctgaatattcatgcacagatgatgctcccagtcaatatgtttgatttttttttctgtttactaatagattttcaaattcactgtgcaaaccaaaattcttaaaaaaaaaaaaaaaggaacttgtgttgaaagcatacattatttctgggcagagttgtcagattaaaatatttatactaaaattattcattgtttatctgaaattctaatttaactgggcactctgtatttttatttactaaatctggcaacccctttctggaccaataaccctacgtagaggaaaggacagaagatagcttctaggggacatgcctgcacctgggagttagaaggaaggaaaaccaaagaaggagccagagaggcagatagaggagtctgcagaaatatggagagagaaccaggtgagtatggaactctataatgaatagtgacacacatgtcagggagaggtggggaccatgaggatgctgggttaagaaaggcctgatcgctatagagtttggcctttgcaatgggggatggtgattctgagaatttctaataggtggttctcgatgaagttggggtggagataaggatgcatgtcggaatcacccagaatatatcaaaattgaaatctgtacaccagaagtagtgcccttcctctcgaaggttggggagaaagacatgtgtgtgtaactcttatcaacccagaactcaagtggtccagttttgggtccatgttatggttactaacgcacaaaataaatttggaaaagaacatacaaatcttttcttgtatttttcttgaaaatcatgaaggaggtctataatttgcaataacgaattcaaaagtccttttcatcttcattctacctccttcacagcctctgctgttgtctgctgtattgctctccagaattttctcagattgcagaaatctccctcctcccgtctatttatttttatcacgtaaatcaacttagcctctctttccagcaaaaaggtccaatcaaattccccaaccctgtcccaaatgaagggtgcggaactgagctggcaattacagactattctttggcagtattctgggaaagggaaaagtatgaattttcttgccctgtccctggtattttgtaaagctttccaagtgacttcgatatctggacccttttgagaactgccgctgttaaccaagacataaagctttttcatttcttgaaaggtcctatttcttggcaaatttctccctgcaaagtgcattcttttttctgcactgaccccagaaacaggtgccattgtgtgggagacagcacctgtcatctgtctcaacaaagtccacaaagtgccctgctaggagcagataacccagaacatctacctgagtctcccagtcaagattcctggtctgactaggaatctaacttcaacaagatcttcctgaaaaaagacgctgagcttggacggtccaaccacctccctacctgggataagccggaggatgcttcttggcttccccaaagctgtcttggtggtgatgctgtggcaaccacaggggagattcacgaggagaaagcctggaagaccagagccctggaagtggggcagccagcccagcgggacattcgtaggggcgaggtgtgatctcagccttcattcagctggcctgggagtcctccaagggtcagagtcacaccttactagtctgacccctcaaggttccttaggactccaggtgttttaagaagattctctgaacccccacccttcccacttcacccctcagccctccctgtggttaagaacccaagtgtgaacaaccccaagtttggtggttagtgaggccttcaagcacaaaccctaatcctgcccttttaaaaaaaatattttggacttaagggaggctgtcctcaattgtttcaaacctatttcagagtacctccactccttcaaacctattttcaaaactcaggaaaacgtcgcctttccagacgaactctagtgacagctccattcaccccttggcatttgactgctaaagatcagctgacccctgcagccagaaggaggagagaaaattgctaaatggggacctcatttccagaggtgatcaccttaaaaaagtctggggccttgtttgccagctttgaaatctcaaagggtaatcttagcttcatgtccccagtgtgaataaaacaaacaataaaaaccacctatgaaatctaaattcaaactttcttgcgacctatgcatcatttttttgcattcacattcccagtcaaaacacagacacacacacacacacacccaacagatatataactgcatgtaaaatatatatagataaaacaatgtcctctgaagaatataaatgttaacacaagtaaaattatcatgaattccataactccttctgtggccttggtccacgttaggttttccattaagagatactattgtcatcagtgagtagcttctcctgttgtattcttctgtgtagcacggggctggagctgtggatatttatgaaccccaggcacccacttcctgttttcgtggctgtccccggtccctgcctctgtgccgttttctctgcctttggccaccagatggcgctctggaaacgagttttctgcatcaaagctcccctgcattactctcaactaggctccctccctcttctctgttcccttccaagccattttccagggtgcagccaaaaggcctttctggagggcacatctgtgtgatcttgggcaagttacctaacgctgtgtgctcagcatcttcaatgtgatggtatggctgtgagtgcgtcatgagaggatgtaggtaaagcacttaggacaaggcctggcatgtggtcgctgttctcattatcctgcgtagagcttttcaaaagcactccacgaccttcaggacaaggtctaaattcttagccatgggaacccaggccactgcatcatctgaggacccctgcttatgtcccctgcccaaccttcctccctacactactgcactgaccctcttgcagcacagccaggagccagcctgcccacttcggggcctttgcccattttgcccctgccaccagctttgcctgtctcctgcctattcaacttcctcgggctcactcctccaggtccgtcaggattcagcgtgtgccctccatgaggagcctcccggacccccaggcctgattccagagccattctactggcctctcgcctttcattttccctgcccccacctgcgactgtcagccccttgccttgttcactgtcaggcacacagggggacgattgcggggcggatagtgagactgtcacgtgggctgccagcagggggcaatgggcacccttgtgtccctgagctcccaggggctcctagtagcttaggcagcaccttgcccacaccaagctgagggagacctgagaaatgggattcctgccaaagaggcaaagaaaaaagtgaagggacaaatgcaaggccatctttgccatttgcgtatgaggcataaaacttggaagataataaacagcaaaatcatagtccttatgtgctgagtgctttctctgagccaggccctgtgctgagtccttaccccaccttgtgagatcgatgacagccctcagagaaggaaacagaggctaggagaggttaagtcccttgtgcagtgtctcagagcttgtgagtggcctgtctgtctccagggccatgctgtaggaggtggccctggattcaggccccttgctatggtttaatgggtttcagtgaaatagttgggaaacctcaggagtgggatatggagttaagggaaagaaaagccagagagaaagggaaggagtgcaggtgactgtcccttagccaccccagtcctgatgaccacaggcctccatgccaataagccctgactagtgccacttgggtccaaacatggcatctctggccccaagggcttagtagcaaacacccatctagggaagctggcgttcattctcatcacctcaaatgcttcatgagcctcagggatcaaccttgaagtgggtataaggctgggagaatgttgggggcagcaaactgaagggcacaataagaagcaataaggccacctcaaagcccacccaagcaaactgctcattcacctccttccttcctgaatttcaccttatgaggaggtgagtggaagatagggtatcccttaaaacatctaaaaggagagttgggggcagcaaaggagatgtgcttcacgggactcttataaacaaaactggggagagaagaattggaggaagggggaaagacatagatgaaagggaggggaaggtgtgggagagggaaacgtataaaagcttccaagaggagtgggaggctgggggttccccagacagagactcagtctggaccagatgcagagaacaatggacttcaaggctggaggggggcagaagggaagcgggaggagagccacacggtcaagttgcacaggttcttgcagcttctggaatcaagaccatgggcaccctcataagtcagtgtgggcagggactgccccagggccaatccaagatccagaggtagccatagggtgtgacaagttgtgcagattacaacactcaccccttgcaataacgtcactgcctgtgactcggggccaggcccaggccaaagcccttcctacatcatttcgtttaatcctcacagtttcctgctgaaagggctactattcttactcccatccccactctacagatgaggtaatggaggcccaggaaagttaagtgacttgtcccagatgacaccgctggtaagttgcaaagtcagaatttgaactcaggcagtttacctctgatggctgctctgttaatcacagctgctttccagtgagacaaaaacgggtgatcagggcagagtcaagacagagaggtaaacaagattgggaaaaagacaggaatgagaggggaacaatgggggaaaagataggaacaaagagagttggggaaggggagagaaacaggaaacatgacttgcccgggaggggcatcagtccacgtgcaagcaggtggaggctcaagttttctgctcacttggtgatgcagaggctccctttccctcagcagccgccttgctgcgtggacagcagcttcccatctggcctgtccccggagccccggcctcatcctcctcagcggcaggccacttagcttcacaggaaatgctctttctctaattggcattgaaactcacagccctcccttttcctgtaggtggggtttccataggaaaaagctgcttctctgtttccccagcctagcaactgtttggcagtcagagtcccacatcctgctcaactgggtcaggtccctcttagaccagctcttgtccatcatttgctgaagtggaccaactagttccccagtagggggtctcccctggcaattcttgatcggcgtttggacatctcagatcgcttccaatgaagatggccttgccttggggtcctgcttgtttcataatcatctaactatgggacaaggttgtgccggcagctctgggggaaggagcacggggctgatcaagccatccaggaaacactggaggacttgtccagccttgaaagaactctagtggtttctgaatctagcccacttggcggtaagcatgatgcaacttctgcaacttctgctggggcttttggggccaggtggctacttatttcttttaggggattgtcaggaggtgaccactctcacggtgaaataccaagtgtcagaggaagtgccatctggtacagtgatcgggaagctgtcccaggaactgggccgggaggagaggcggaggcaagctggggctgccttccaggtgttgcagctgcctcaggcgctccccattcaggtggactctgaggaaggcttgctcagcacaggcaggcggctggatcgagagcagctgtgccgacagtgggatccctgcctggtttcctttgatgtgcttgccacaggggatttggctctgatccatgtggagatccaagtgctggacatcaatgaccaccagccacggtttcccaaaggcgagcaggagctggaaatctctgagagcgcctctctgcgaacccggatccccctggacagagctcttgacccagacacaggccctaacaccctgcacacctacactctgtctcccagtgagcactttgccttggatgtcattgtgggccctgatgagaccaaacatgcagaactcatagtggtgaaggagctggacagggaaatccattcattttttgatctggtgttaactgcctatgacaatgggaacccccccaagtcaggtaccagcttggtcaaggtcaacgtcttggactccaatgacaatagccctgcgtttgctgagagttcactggcactggaaatccaagaagatgctgcacctggtacgcttctcataaaactgaccgccacagaccctgaccaaggccccaatggggaggtggagttcttcctcagtaagcacatgcctccagaggtgctggacaccttcagtattgatgccaagacaggccaggtcattctgcgtcgacctctagactatgaaaagaaccctgcctacgaggtggatgttcaggcaagggacctgggtcccaatcctatcccagcccattgcaaagttctcatcaaggttctggatgtcaatgacaacatcccaagcatccacgtcacatgggcctcccagccatcactggtgtcagaagctcttcccaaggacagttttattgctcttgtcatggcagatgacttggattcaggacacaatggtttggtccactgctggctgagccaagagctgggccacttcaggctgaaaagaactaatggcaacacatacatgttgctaaccaatgccacactggacagagagcagtggcccaaatataccctcactctgttagcccaagaccaaggactccagcccttatcagccaagaaacagctcagcattcagatcagtgacatcaacgacaatgcacctgtgtttgagaaaagcaggtatgaagtctccacgcgggaaaacaacttaccctctcttcacctcattaccatcaaggctcatgatgcagacttgggcattaatggaaaagtctcataccgcatccaggactccccagttgctcacttagtagctattgactccaacacaggagaggtcactgctcagaggtcactgaactatgaagagatggccggctttgagttccaggtgatcgcagaggacagcgggcaacccatgcttgcatccagtgtctctgtgtgggtcagcctcttggatgccaatgataatgccccagaggtggtccagcctgtgctcagcgatggaaaagccagcctctccgtgcttgtgaatgcctccacaggccacctgctggtgcccatcgagactcccaatggcttgggcccagcgggcactgacacacctccactggccactcacagctcccggccattccttttgacaaccattgtggcaagagatgcagactcgggggcaaatggagagcccctctacagcatccgcagtggaaatgaagcccacctcttcatcctcaaccctcatacggggcagctgttcgtcaatgtcaccaatgccagcagcctcattgggagtgagtgggagctggagatagtagtagaggaccagggaagcccccccttacagacccgagccctgttgagggtcatgtttgtcaccagtgtggaccacctgagggactcagcccgcaagcctggggccttgagcatgtcgatgctgacggtgatctgcctggctgtactgttgggcatcttcgggttgatcctggctttgttcatgtccatctgccggacagaaaagaaggacaacagggcctacaactgtcgggaggccgagtccacctaccgccagcagcccaagaggccccagaaacacattcagaaggcagacatccacctcgtgcctgtgctcaggggtcaggcaggtgagccttgtgaagtcgggcagtcccacaaagatgtggacaaggaggcgatgatggaagcaggctgggacccctgcctgcaggcccccttccacctcaccccgaccctgtacaggacgctgcgtaatcaaggcaaccagggagcaccggcggagagccgagaggtgctgcaagacacggtcaacctccttttcaaccatcccaggcagaggaatgcctcccgggagaacctgaaccttcccgagccccagcctgccacaggccagccacgttccaggcctctgaaggttgcaggcagccccacagggaggctggctggagaccagggcagtgaggaagccccacagaggccaccagcctcctctgcaaccctgagacggcagcgacatctcaatggcaaagtgtcccctgagaaagaatcagggccccgtcagatcctgcggagcctggtccggctgtctgtggctgccttcgccgagcggaaccccgtggaggagctcactgtggattctcctcctgttcaggtacctggggcatgggcagctctttctctggtcactcctggaccaccagaaacagaatcagacacccccataacctgcacagtcctcatgtttcttccttaggctcacctggcactttctgatgctttgggtagcaatggttcagggcacagctttgtgatcggctgaacttagctgtgtgatcctaggcaaattccttactgtctctgggccttagttgtcttatctgtaaattagagatgatagtaagagtatctaccttgtagagttgtccagaggatttgatcaattaattcctataaggcatttatggtgcctcacaagtagacacacagcaaaggtagtgattaagagtataagctttgggcaaggttacctgggttcaaatcctggctccacttcttcctagctctgaaccacagaggaagttaacatctctgttgaagatgtctatgttaacaccacccatcttctaagtttgcttgaaaataaaatgagtgaatgctcttaatgcctgtagaacagtgtccatctgacatgtagtcagtactcagcaaattattactattacttcctcaaagcacttctccatctccactatttgcctgccttcatctcatctgggcaacttccctatgaagaaggccagcataggcatcattgcacccaaacccaggtgttgcaaataatttgtctgagaccccagagctttccggctgggcctgcggatggggcagtggaaattaccttagaacttagcagttaggcagattggagttcagatttcaacacagccacttaggagctgggcaaccttgtacacgtccttcaacatctgagcctcattttcctcatctataaaatggggattatgagaccaaacttaccctgtgtctgcctccttagagtgtgagggctaaatgggatgatgagtgtcaaaatgtctaaggacctgggattttctagacacaagtggggaaacctttccagggatgatgcctgatctcacacccatatctgcttctgtgaacctcagatattttcactgtgccaggctgcccgcccaaggcacacttctgtttggccaggctatcagctcaggtttcctgtgaagcaaaacacagtttccttccctattccctcctcaggaagccacctggagatggcccagctgttctcttaggaatcttgccctgggatcggtgcagacccagaggctgccagagccaaccattcatttatccttccattcatccatccatttatcaggtgctttttggataaactctgtgctgaacactgtgctggctgctggtagacaggacaacagagaataggccctgtgggtgaccccgctgaggtgacagaaagaaaggtgcccatttgggatcaacttgagggtaacaggcaacttgcccaatgctcatatctttccagcagcacatagagtcagatctgagtgtaaattccagccctcccactaatttctgtgtagcccctgtgagcctccaattccacctctgcaaaatggccataatggtatctcttcccttaggctgttgtaagaattaaatgagattatacaccactggacaatcactgtttatcatgggatttattaataagctcatttaaacctcgtaacccaccatatgacctgggtctcattaatatgcactttttttactgttgaggaaactgagtaattttctcagaatgcttaagtaattcctcaaggccacactgatgggaactggggcagggacatatttaggatttgaacctgagtctgactgattcgagtctgggctgtaactgtgaagtgaccctgaatgacagtgctcagcatagggcttggcatgtagggagtcatgtcatgaaaaggcagctgtcacttgagcaaagggtggcaggtatcttaggagggcttctgggcctgggctgggaggtgggatcttccctgaggcaggcaataagaccccgagtctcccaagtcacagtgaaaccccagtcctggccatctcctcttccaactctcctctgaggttgacacttcctacctcaagctgcacagaggggcatggtggacctcctaagggaggaatggggatggacccaggaggaggaagcagtgtttctaggaataacagtgcaaactccttgcttttgcagaggatttttaactttttcaaaccatttaatcgtgacaacactgagaagaaggcgtgtgggtatttctagccccctattagagatgaggaaactgaggtctgaggaggtctaaaaacctgtctaatgctatacaataagctacaaatggccttgaactagaactccgcctcccaggacttgatctgttgcctctcccttccaatggcctatagggtctggccgtgatatctcacccatcccctactccattctccagtctttctgaaactctaccttctcttccagcctttcagtgtgcagtgcttctgcctgaaaacctccccaccccttgcccagaccttcattctctttccacttgccaaactcctatggagcctttgggtctcagcagaggtgcctggcatgtggtatgaactcagtaacatctgttgaaatgaatgaattaacttaatccagtaccacccccctgcttcctccctgctccaagttgggttaggggccctctgctcccacagagcccccattctttccctatgcccagaggcaacccctgtggtaaacactcttcgctgccctgatctccagccagcctccctgtggggctgcctgcaaccttcacatcctgacagatgtgaccacgctgagggcagcagctggggctccttctccttcttactcccatagcaaccaccctgcctggcacttattaagtacttgttgaatgaatgaaaagatgaataaatgaataattcaatgacaccattttgccttctctacaacatagtcttacatctggtcaacaaaaattggccgaatgtccaaatactgctaaaggggaacataactctttaagataatcttcatcaaagtaacaacaaattgatggtcaggaaatgcctttttcaccccatctctcagagtccaccaggtgcaggacactcggaggaatgagtatgaggggacaggcccagagggtcctcttgctctccttcagacactcaggtcctggggtgacatgacagagcaggggacctgcccgcccaggtccctgtctctgtcactgggcaccatcccccatctgatcacacaaacatctgaagccagaggaaagactataaaaagaaagcaccggcattgagggaaacatccggcgaggaagtgtgctgagtcggaattgtgtggagtcaacagatttttcctgtcttgtagcaaatctcccagctgctgtccttgctgcatcagggccaattccagcccaaaccaaaccaccgaggaaataagtacttggccaagccaggaggcagcaggtaagcagcacgtagcccccacaggcatctcaaggcccctactggccgcccctcatcactgcagcctctgtgagtgaggaagggtgagcacaagtcagacaccggtcccctgggagggtgcagcagacagcatccatcctcccaccctgggagttttggtcgtgaggtgactgtattgatagtatttacaggacctagaagtcctgtaggactaactagctgtattgataaataccatcaatacagtagtcaccaaggaaatgacctcatccctgagactgtatttgtcccaattacagaggtgctgtaaattgcctttatatctgtaactgtcacctctctttctcttgacccataaacataatatgaaggctccccactaaagacatggctgtgtgggcgtgtccctttgggcaatcctagaattgaagacaccttaagagaacatcaaatccaacttcttgttacggatggagaaactgaggctctgagtagcttgattcaaaccagctatgtaaacttgagcaagtgagataatctctcaggaccttggtttccccatctgtaaaacagaacccatcccatttcccttgtggtaatggggtgaggatttcataatcaattgtattctaagcccatagcccagcaactgacacatagtaggtgcacacacaatgctaactcctttccccttgaagcaattttgctaaaggtcatgtaacaagtgagtggcaaagctgggaccagctctcagagttcctgactcctgcatcagtgctcttgccctgacaacttacagcttctttttacaaggatggtgagaggtagtgtactgcaggggtgagtccacacactccagggtcacactggcctggatttgaagtcttgctttatctcttaatctatggggaccttgggcaacctgcaaaatcttcacaagtcttggttttctcatttgtacaatgggttgattaaagagaaccccttacaggtgtggaaggtttgaataagacggtgcatggaccttgcatcgcataaggcctgactcagactcactgctcaaaaacattagttgtcattatcacccacacacttgccacctccctgctcatttcttcacagcagcagaaatggctacaggggaacaaaaccaaacaaacagaaggactcctacgccttaaaacccctcaggtagccttcccacctgctgggatgagggtccaggcctgtccccaagtgtctatgggagagagggaccaggatggagacagcaggagactggggtgcaggtagcagattctgagcagctttaggctgcctcactccggctgcaccctccataggtctgctcttctggcctaggatttggcctaaagtagggaggaaggaatgttcagtcactcattcagcacccactgtgtgccacgccctgttctgagcatcagtaagggagcaggcaatgagacaggcaaaaatcccagccttctcctacggacggatggacggctgcactggctccactgaggtttactaaactcctcctgtgtgccaggcaatacctagacatgggaggggcaacgagaatgatatcaatgaactcatcttcacttctgtctgctgagtgcttacaatgtgccaggctctgtggtaggcccctctacctacaacgtctcattttaccctcacaacaccctgtgaggtgggtgttatttattactctcattttacaaatgaggaaacagagtaagaggagggcgggaatcacatgatccaggttggtctgactctagaggccaggctgtgtatccagttccctatatgggtaaatgaatgcaaacatgaatgggcatgaatgaatgaatctatatgggaatgtccctacccatggactggttggtttcttttgcaggagtgcaatcccagacacagatggcccaagtgcaagggctggaggccagacagacccagaacaggaggaagggcctttggatcctgaagaggacctctctgtgaagcaactgctagaagaagagctgtcaagtctgctggaccccagcacaggtagggaccccctggagttaccttgaccctgcttctgccccatggtgtccaagcttcagagttctgctccaatccactgcagggaaaagccattctgtttctgtggaggaacatcagcaacatttaacttcccttgcacccttgcagcctcagaaagtattttaaagaccaggttctactttgattttttttttttttagacagggtcttgctctgtcacccaggctggagtacagtggcatgatcacagcatgatcacagctcactgcagcattgacctcctgggctcaggtgatcctcccaccttagcctcccaagtagctgggactacaggtgagcaccaccaccacatcccgctaatttttgatttttttttttttttgtagggacagggattcacctgttgcccaggctggtctccaactcctgggctcaagcaatccacccacctcagcctcccaaagtgctggcattagaggcatgagccacctgtaatcaaagccaaagtaggcacctggcctacgttgattattcattttaaaaatcattgtaaaaatagtgaacatgccaggcactggggaagcatcttatgtacatgatctcatttaagcctccaaccaatcccctcaagtagataattattacctcgaatttacagatgagcaaaccgaggctcaaaaaaatcaagtagccttcccagagtcacccagctcaccagtgattgaaggggagccaaaccctgagccagcctgtcctctatggacttcttaccactcattggcgtttggtgtgtttcaataccctttcaccctgggaaccaggtcaactctttaatatgctaacaattgcaatgaaaacaaaaagatttttttgcagagaaataattagctttgacaataaatacataatgtgaacttgaattcaagaatctgctagcgccccccgcccctgctgtggacattggtcagcctcctttggtaacacaccccacggcagaccatccctgtgctgtggttttctatctatgtagttaacactgtttctcatgtgctccatctggctgcagacacactgaaggtatggcagggacttctctttccttggttaaaaaatattgctaatgtttattgggcactttctatgtgctaggccctgttctaagtacctcatgagaaaatcttatgagataggggctattcattatccccatttcataaaggaaaaatgtgagggtcaggggtgtttcataatttgcccaaggttgtgcagctcgtggatggttattaacacaggcagtgtggctgtaaattttagagctgtgttgttcaatgaggtagtcactagcgacaagtggctatttaaatttaagtgaattaaaattaaataaaataaaatattcagttctgccataccagccacatttcaagtgctcactggctatgtatggctgggggatatagctttggattctatctctattggtcagctctggctggaacctgtggggtcagttcccacacggtcctgcttgcccacagcacaaagggtactgaacacaggggcactgtaaggagttgggggtaaggaaagaggaggggcagaaaagcctttgctgctcacaggctggactgccatggggagactgggatcttgctgcttcagatctgcttcagattgcttcagatttgctgcttcggattcagattgccccacttgagaatgcgaccatgataatgttttcctggttgcatgaactcactgaacctcacaacaactccacaagatacgtactactgtctcaccacttcctaagaggaaacgggagcttagagaggttaagccattttcccaaggtcacacacactgcccctaagctgcaaagctgggaaatcaaacccaggttggcccagtgcccacctgcagctagaactggtgggtgatgctgggtgaattcagcatcacttttcagctgttctcatacgccccagtgattttctcattccaagcttcactccttctgaagctctctgacccatgtaaaaggaaaggcctgattctataacaccaagagttccatccaaccttatagcctcagagtctcaaggaactaaggtttgcatttcccaaatgttgaaggttaatatttatgttcattcaatcattcaacaaacattcactaagtcccaggcatggggctaaacccaaggatccagtagggcaggagcccaacagtgtctcacaagctgcacagtccagaatgcagggcagagattcatttatcagtacaataaatgactacacatacttcggaataacttgttcaaattacataagtaatatttggttaatatagaaaaacatagataaacaaaaagagaatggattgcttctgcccagaggtaatttcagttaacattttgacatatctccttccagtctttcattctgtgcgcagattttaaagtaactcaccttaggcaaacattagataaatgttaagtagatgttgtgcttttttttttttttttaatttttttaagacactgtttcttaactccaagacttataatttctttcctctggcttggtctctaccatcaggcctgggactggtttcttgaaacaggattttctgttaaagggatgggaaaagaagggtgactgtggcattgcaaatgtaggggccagttttttgaattcccagccaccaggctgacttgccaccgccactaccccagtccctctcctgggacagcccttggacaggcagcccctccccagcctgactctaggccagggttccagcagcttgagagatgatgttgaggaggatttgaggatctggggccaggtggtaacagtgcagaccttaggtagcctgggaattccagaggccccagctgggcacctggagtatccagggggaggaagggccagctcttcagagagagggtagggtggtgccatcataggcttcaaagtcatcagacctggcatcaaattgaggtttggttgcctagcagatgtgtgacttaacttctttcagcttcagtttcctcatccatcaaatgggaataatactcttgacctcaaaggtttgtgaagttaaatgaaataattatgtaaattgctcatcacgtaagtacctaatatgtggtccctattattatggatgggaaccaaaaaaggcagcgctaagtcaggaactccagccttggcaaagaaaagggattttccctccatcctcagctagtggagccctgaggggagaaggcgccctgaggggtaaaaatcctggatgaaatatagaagacactgagactggaaacacacgcccagattgggaaattcttcagaaatcggcaaatttctacttctaggccttctcctcctcctgctgcttccccacttggttcgcccgggtgtgtcagtcgactgctttcctacccagggagcttccctgtcagggcctcttccttcctgtccccgtctggccactctgtctgtcccctgccatttcccttccctcctttgtccactgcccttgccctgctcattgccccaccctcacccgcctggcccttcccggagcctggccctcactgtgtcccctccttcccccacaggtctggccctggaccggctgagcgcccctgacccggcctggatggcgagactctctttgcccctcaccaccaactaccgtgacaatgtgatctccccggatgctgcagccacggaggagccaaggaccttccagacgttcggcaaggcagaggcaccagagctgagcccaacaggcacgaggctggccagcacctttgtctcggagatgagctcactgctggagatgctgctggaacagcgctccagcatgcccgtggaggccgcctccgaggcgctgcggcggctctcggtctgcgggaggaccctcagtttagacttggccaccagtgcagcctcaggcatgaaagtgcaaggggacccaggtggaaagacggggactgagggcaagagcagaggcagcagcagcagcagcaggtgcctgtgaacatacctcagacgcctctggatccaagaaccaggggcctgaggatctgtggacaagagctggtttctaaaatcttgtaactcactagctagcggcggcctgagaactttagggtgactgatgctacccccacagaggaggcaagagccccaggactaacagctgactgaccaaagcagccccttgtaagcagctctgagtcttttggaggacagggacggtttgtggctgagataagtgtttcctggcaaaacatatgtggagcacaaagggtcagtcctctggcagaacagatgccacggagtatcacaggcaggaaagggtggccttcttgggtagcaggagtcagggggctgtaccctgggggtgccaggaaatgctctctgacctatcaataaaggaaaagcagtgattcattctcctgtttgcacctgtcaagagggagaagggaagagtaatagagtggggtttatttgtcatccaatccctggcaagaccgtcacactgggcctggacaggagtggggcaccttggtacacagtaacaggtacacaaatagcagacatgtgtgctcttacccctgttcccctatcactatgttaccaactaatctgcttctcagctgcaaaattagagctaagaaagcacaactatttataaagcatttacatgagccaggcaccaggctttacaataggatttaatttgattttccaacaatcctgggagggaggtattattaaccccatttcacagacacggatatggggcttggagaggtcaagtggctttcccaagatcaagcagggagtagagccagagccaggatttggacccaggctgggccaacaccaaaacccatgccttcaaccttgacaccagcctgccaaccaaagacaggagaagggaaggccctggaggtgagctgttggcagcagtgaacttgctcgagctccctcgggaaacactgttgaaaggaacatttccaaagccatccaggcccgcacctctctacattcccaagcacaaggcaaaaggaacttggatctgaactgatgcagacccgttctttcccaacaagtagctcttcaagtgcggatgtcatctttcccaaccccatttgcgggtaaaccttcctctgtcattgcaggttcaggcttgtgaatcccagaggacgctgtgtgaactgggtctatgaaatcggcactgacagcagccagccttccaggggatgggggagggaagaaacagcagatattttcccaaggttcagctttataattttcttggaaatttccgagcagccaatcagctagcttctaaatagtgtgggtctctcctggacactgcaggcaaaagttcctctatctgctcctctttttgtcctttcttggagctcagtgctcatgttcactgttcaagaaatgggccaatcatactcccaactagggataatgtgcatcata

SEQ ID NO: 6 - DNA, 15 bp, Homo sapiens
gccggcagctctggg

SEQ ID NO: 7 - DNA, 22 bp, Homo sapiens
ctgagcaagccttcctcagagt

SEQ ID NO: 8 - DNA, 24 bp, Homo sapiens
cactctcacggtgaaataccaagt

SEQ ID NO: 9 - DNA, 17 bp, Homo sapiens
ggatccgggttcgcaga

SEQ ID NO: 10 - DNA, 20 bp, Homo sapiens
cctttgatgtgcttgccaca

SEQ ID NO: 11 - DNA, 22 bp, Homo sapiens
ttggagtccaagacgttgacct

SEQ ID NO: 12 - DNA, 19 bp, Homo sapiens
tgtcattgtgggccctgat

SEQ ID NO: 13 - DNA, 19 bp, Homo sapiens
tcgacgcagaatgacctgg

SEQ ID NO: 14 - DNA, 22 bp, Homo sapiens
cgcttctcataaaactgaccgc

SEQ ID NO: 15 - DNA, 24 bp, Homo sapiens
ttgtgtcctcaatccaagtcatct

SEQ ID NO: 16 - DNA, 21 bp, Homo sapiens
cagcccattgcaaagttctca

SEQ ID NO: 17 - DNA, 22 bp, Homo sapiens
ggtgcattgtcgttgatgtcac

SEQ ID NO: 18 - DNA, 21 bp, Homo sapiens
caggacacaatggtttggtcc

SEQ ID NO: 19 - DNA, 26 bp, Homo sapiens
caatagctactaagtgagcaactggg

SEQ ID NO: 20 - DNA, 21 bp, Homo sapiens
tgacatcaacgacaatgcacc

SEQ ID NO: 21 - DNA, 19 bp, Homo sapiens
acggagaggctggcttttc

SEQ ID NO: 22 - DNA, 28 bp, Homo sapiens
gctcacttagtagctattgactccaaca

SEQ ID NO: 23 - DNA, 16 bp, Homo sapiens
tgcccccgagtctgca

SEQ ID NO: 24 - DNA, 20 bp, Homo sapiens
ctctccgtgcttgtgaatgc

SEQ ID NO: 25 - DNA, 20 bp, Homo sapiens
ctgagtccctcaggtggtcc

SEQ ID NO: 26 - DNA, 22 bp, Homo sapiens
ttcatcctcaaccctcatacgg

SEQ ID NO: 27 - DNA, 19 bp, Homo sapiens
gcacgaggtggatgtctgc

SEQ ID NO: 28 - DNA, 21 bp, Homo sapiens
tgtactgttgggcatcttcgg

SEQ ID NO: 29 - DNA, 21 bp, Homo sapiens
gaggttgaccgtgtcttgcag

SEQ ID NO: 30 - DNA, 20 bp, Homo sapiens
gggcagtcccacaaagatgt

SEQ ID NO: 31 - DNA, 19 bp, Homo sapiens
tgagatgtcgctgccgtct

SEQ ID NO: 32 - DNA, 21 bp, Homo sapiens
gagaacctgaaccttcccgag

SEQ ID NO: 33 - DNA, 21 bp, Homo sapiens
gaggactgtgcaggttatggg

SEQ ID NO: 34 - DNA, 18 bp, Homo sapiens
gtccggctgtctgtggct

SEQ ID NO: 35 - DNA, 23 bp, Homo sapiens
ggcaccataaatgccttatagga

SEQ ID NO: 36 - DNA, 25 bp, Homo sapiens
cttagctgtgtgatcctaggcaaat

SEQ ID NO: 37 - DNA, 28 bp, Homo sapiens
tgagtactgactacatgtcagatggaca

SEQ ID NO: 38 - DNA, 25 bp, Homo sapiens
acttcttcctagctctgaaccacag

SEQ ID NO: 39 - DNA, 25 bp, Homo sapiens
aactccaatctgcctaactgctaag

SEQ ID NO: 40 - DNA, 21 bp, Homo sapiens
atgaagaaggccagcataggc

SEQ ID NO: 41 - DNA, 22 bp, Homo sapiens
gatatgggtgtgagatcaggca

SEQ ID NO: 42 - DNA, 20 bp, Homo sapiens
accatcccccatctgatcac

SEQ ID NO: 43 - DNA, 19 bp, Homo sapiens
ggatgctgtctgctgcacc

SEQ ID NO: 44 - DNA, 21 bp, Homo sapiens
ccaggctgtgtatccagttcc

SEQ ID NO: 45 - DNA, 21 bp, Homo sapiens
cctccacagaaacagaatggc

SEQ ID NO: 46 - DNA, 18 bp, Homo sapiens
cccttgccctgctcattg

SEQ ID NO: 47 - DNA, 17 bp, Homo sapiens
cctcccgcagaccgaga

SEQ ID NO: 48 - DNA, 18 bp, Homo sapiens
ctgagcccaacaggcacg

SEQ ID NO: 49 - DNA, 23 bp, Homo sapiens
agtcaccctaaagttctcaggcc

SEQ ID NO: 50 - DNA, 18 bp, Homo sapiens
cccaggtggaaagacggg

SEQ ID NO: 51 - DNA, 21 bp, Homo sapiens
tcctgcctgtgatactccgtg

SEQ ID NO: 52 - DNA, 21 bp, Homo sapiens
aagagccccaggactaacagc

SEQ ID NO: 53 - DNA, 19 bp, Homo sapiens
ctgtccaggcccagtgtga

SEQ ID NO: 54 - DNA, 19 bp, Homo sapiens
gggtgccaggaaatgctct

SEQ ID NO: 55 - DNA, 23 bp, Homo sapiens
gggttaataatacctccctccca

SEQ ID NO: 56 - DNA, 27 bp, Homo sapiens
atgttaccaactaatctgcttctcagc

SEQ ID NO: 57 - DNA, 21 bp, Homo sapiens
tcctttcaacagtgtttcccg

SEQ ID NO: 58 - DNA, 19 bp, Homo sapiens
ctgggccaacaccaaaacc

SEQ ID NO: 59 - DNA, 20 bp, Homo sapiens
gaaggctggctgctgtcagt

SEQ ID NO: 60 - DNA, 23 bp, Homo sapiens
aagtagctcttcaagtgcggatg

SEQ ID NO: 61 - DNA, 25 bp, Homo sapiens
tatgatgcacattatccctagttgg

SEQ ID NO: 62 - DNA, 22 bp, Homo sapiens
ggtgcctgtgaacatacctcag

SEQ ID NO: 63 - DNA, 24 bp, Homo sapiens
gataggtcagagagcatttcctgg

SEQ ID NO: 64 - DNA, 22 bp, Homo sapiens
tgactcctgctacccaagaagg

SEQ ID NO: 65 - DNA, 23 bp, Homo sapiens
cttgccttaggcttatctccctt

SEQ ID NO: 66 - DNA, 22 bp, Homo sapiens
gcctcggaatgtcagctacttt

SEQ ID NO: 67 - DNA, 19 bp, Homo sapiens
ggtcatctggtgcctttgg

SEQ ID NO: 68 - DNA, 22 bp, Homo sapiens
ccagcctaacaatgctctcctt

SEQ ID NO: 69 - DNA, 45 bp, Homo sapiens
gaagatcttcggaattccatcatgatgcaacttctgcaacttctg

SEQ ID NO: 70 - DNA, 55 bp, Homo sapiens
aagatcttcggtacctcaatggtgatggtgatggtgcaggcacctgctgctgctg